(19) United States
(12) Patent Application Publication
(10) Pub. No.: US 2023/0045289 A1
(43) Pub. Date: Feb. 9, 2023

(54) CLEANING COMPOSITIONS COMPRISING DISPERSINS IX

(71) Applicant: Henkel AG & Co. KGaA, Duesseldorf (DE)

(72) Inventors: Mirko WEIDE, Duesseldorf (DE); Susanne WIELAND, Zons/Dormagen (DE); Brian LAUFS, Juechen (DE); Michael KANDZIA, Solingen (DE); Ulrike DENGUTH, Oer-Erkenschwick (DE); Christian DEGERING, Erkrath (DE); Rune Nygaard MONRAD, Bagsvaerd (DK); Rebecca VEJBORG, Bagsvaerd (DK); Jesper SALOMON, Bagsvaerd (DK); Dorotea Raventos SEGURA, Bagsvaerd (DK)

(21) Appl. No.: 17/786,557

(22) PCT Filed: Dec. 7, 2020
(86) PCT No.: PCT/EP2020/084952

§ 371 Date: Jun. 17, 2022

(30) Foreign Application Priority Data
Dec. 20, 2019 (DE) 10 2019 135 361.0

(51) Int. Cl. C11D 3/386; C12N 9/24; C11D 3/22; C11D 3/36; C11D 3/12; C11D 3/395; C12N 9/26

(52) U.S. Cl. CPC C11D 3/38636; C12N 9/2494; C11D 3/222; C11D 3/361; C11D 3/128; C11D 3/3956; C12N 9/2411; C12Y 302/01052

(57) ABSTRACT

Non-limiting cleaning compositions may include a mix of enzymes including dispersins and specific amylases or mannanases. Said compositions may be used in cleaning processes and/or for cleaning of items, such as textiles, as well as methods of using said compositions for removal or reduction of organic stains.

### CROSS-REFERENCE TO RELATED APPLICATIONS

The present application is a national stage entry according to 35 U.S.C. § 371 of PCT application No.: PCT/EP2020/084952 filed on Dec. 7, 2020; which claims priority to German patent application 10 2019 135 361.0, filed on Dec. 20, 2019; all of which are incorporated herein by reference in their entirety and for all purposes.

### REFERENCE TO A SEQUENCE LISTING SUBMITTED VIA EFS-WEB

The content of the ASCII text file of the sequence listing named “2019P00545WOUS_P83441_seqlist_ST25_final”, which is 117 kb in size was created on Dec. 20, 2019. The sequence listing was corrected on Jun. 15, 2022 to correctly define the source of the genetic material; the corrected sequence listing is electronically submitted via EFS-Web herewith; the original sequence listing and the corrected sequence listing are incorporated by reference in their entirety.

### TECHNICAL FIELD

The present invention relates to compositions such as cleaning compositions comprising a mix of enzymes, as defined herein. The invention further relates, to the use of compositions comprising such enzymes in cleaning processes and/or for cleaning of items such as textiles as well as methods of using said compositions for removal or reduction of organic stains.

### BACKGROUND

Enzymes have been used in detergents for decades. Usually a cocktail of various enzymes is added to detergent compositions. The enzyme cocktail often comprises various enzymes, each targeting a specific substrate e.g. amylases are active towards starch stains, proteases on protein stains and so forth. Textiles surface and hard surfaces, such as dishes or the inner space of a laundry machine enduring several wash cycles, become soiled with many different types of soiling which may compose of proteins, grease, starch etc. One type of soiling may be organic matter, such as polysaccharides e.g. PNAG (poly-N-acetylglucosamine) and proteins. Some stains may be sticky or gluing, which when present on textile, attracts soils and may cause redeposition or back-staining of soil resulting in a greying of the textile. Additionally, organic stains often cause malodor issue as various malodor molecules can be adhered by the polysaccharides and proteins. There is still a need for cleaning compositions, which effectively prevent, reduce or remove components of organic stains such as polysaccharides. The present invention provides new compositions fulfilling such need.

### SUMMARY

The invention relates to a cleaning composition comprising a dispersin and at least one carbohydrase and, optionally, at least one cleaning component, wherein the carbohydrase is an amylase and/or a mannanase selected from the group consisting of:

(I) a mannanase variant, or a recombinant polypeptide or an active fragment thereof comprising an amino acid sequence comprising two or more modifications selected from:
(1) (a) one or more substitutions at one or more positions selected from 1, 2, 3, 4, 6, 10, 19, 28, 30, 38, 59, 60, 61, 62, 63, 66, 67, 68, 70, 71, 74, 75, 78, 80, 82, 93, 97, 103, 111, 124, 129, 131, 135, 136, 139, 143, 150, 167, 168, 184, 213, 214, 217, 225, 228, 235, 242, 244, 258, 259, 261, 283, and 284, and (b) an insertion at position 298; or
(2) one or more substitutions at one or more positions selected from 19, 38, 59, 67, 68, 71, 74, 97, 129, 167, 168, 184, 225, 228, 235, 242, 244, 258, and 261;
wherein the amino acid positions of the variant or recombinant polypeptide or active fragment thereof are numbered by correspondence with the amino acid sequence of SEQ ID NO: 24; and/or

(II) a mannanase variant comprising an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence according to SEQ ID NO:39, 40 or 41; and/or

(III) a variant of a parent alpha-amylase comprising:

a mutation at an amino acid residue corresponding to R375, and optionally S360; and at least one mutation, and optionally at least two mutations, at an amino acid residue, or residues, corresponding to an amino acid residue selected from the group consisting of N126, F153, T180, E187, and I203; wherein the variant alpha-amylase or the parent alpha-amylase has at least 60%, at least 70%, at least 80%, at least 85%, 86%, 87%, 88%, 89%, 90%, 91%, 92%, 93%, 94%, 95%, 96%, 97%, 98%, or 99% amino acid sequence identity relative to SEQ ID NO: 36, which is used for numbering; and wherein the variant preferably has increased low pH stability and/or starch liquifaction activity, compared to the parent alpha-amylase or a reference alpha-amylase differing from the variant α-amylase only by the absence of the mutations; and/or

(IV) a variant of a parent alpha-amylase comprising:

i) a mutation at an amino acid residue E187 using SEQ ID NO: 36 for numbering; and at least one mutation at an amino acid residue selected from the group consisting of N126, Y150, F153, L171, T180, and I203 using SEQ ID NO:36 for numbering; wherein the variant alpha-amylase has at least 70% amino acid sequence identity to SEQ ID NO:36; or

ii) a mutation at an amino acid residue E186 using SEQ ID NO:37 for numbering; and at least one mutation at an amino acid residue selected from the group consisting of N125, Y149, F152, L170, D179, and L202 using SEQ ID NO:37 for numbering; wherein the variant alpha-amylase has at least 70% amino acid sequence identity to SEQ ID NO: 37; or

iii) a mutation at an amino acid residue E189 using SEQ ID NO:38 for numbering; and at least one mutation at an amino acid residue selected from the group consisting of N128, Y152, F155, L173, T182, and L205 using SEQ ID NO:38 for numbering; wherein the variant alpha-amylase has at least 70% amino acid sequence identity to SEQ ID NO:38;

wherein the variant preferably has increased thermostability, detergent stability, starch liquefaction activity, and/or cleaning performance compared to the parent alpha-amylase or a reference alpha-amylase differing from the variant alpha-amylase only by the absence of the mutations.

In various embodiments, the cleaning composition of the invention that comprises a dispersin and at least one carbohydrase, wherein the carbohydrase is an amylase or a mannanase,

(a) is a solid, preferably granular, laundry detergent composition and further comprises

(a1) at least one zeolite builder, preferably in an amount of 10 to 50 wt.-%, more preferably 20-30 wt.-%;

(a2) at least one phosphonate builder, preferably in an amount of 0.1 to 5 wt.-%, more preferably 0.4 to 1.5 wt.-%;

(a3) at least one further enzyme, preferably a cellulase, in an amount of active enzyme of 100 to 5000 ppb, more preferably 1000 to 2000 ppb; and

(a4) at least one polymer, preferably a polyvinylpyrrolidon polymer, preferably in an amount of 0.01 to 1 wt.-%, more preferably 0.1 to 0.3 wt.-%; or

(b) is a solid laundry detergent composition and further comprises

(b1) at least one silicate builder, preferably in an amount of 2 to 20 wt.-%, more preferably 5-10 wt.-%;

(b2) optionally carboxymethylcellulose, preferably in an amount of 0.1 to 10 wt.-%, more preferably 0.1 to 4 wt.-%;

(b3) at least one further enzyme, preferably a cellulase, preferably in an amount of active enzyme of 0.1 to 100 ppm, more preferably 0.1 to 10 ppm;

(b4) optionally at least one soil release polymer, preferably a polyvinylpyrrolidon polymer, in an amount of 0.1 to 3 wt.-%, more preferably 0.1 to 1.0 wt.-%; and

(b5) at least one bleaching system, comprising a bleaching agent, a bleach activator and a bleach catalyst, preferably in an amount of 0.1 to 50 wt.-%, more preferably 0.1 to 30 wt.-%; or

(c) is liquid laundry detergent composition and further comprises

(c1) at least one surfactant, preferably nonionic surfactant, preferably in an amount of 1 to 20 wt.-%, preferably 3 to 15 wt.-%;

(c2) optionally at least one phosphonate builder, preferably in an amount of 0.1 to 3 wt.-%, more preferably 0.25 to 1.5 wt.-%

(c3) optionally at least at least one further enzyme, preferably a cellulase, preferably in an amount of enzyme composition of 0.001 to 1 wt.-%, more preferably 0.001 to 0.6 wt.-%; and

(c4) optionally at least one organic solvent, preferably glycerol, preferably in an amount of 0.1 to 10 wt.-%, more preferably 0.1 to 5 wt.-%; or

(d) is a liquid laundry detergent in unit dose form, preferably a pouch comprising a water-soluble film, and further comprises

(d1) water in an amount of up to 20 wt.-%, preferably 5 to 15 wt.-%;

(d2) optionally at least one bittering agent, preferably Benzyldiethyl(2,6-xylylcarbamoyl)-methylammoniumbenzoate, preferably in an amount of 0.00001 to 0.04 wt.-%;

(d3) optionally at least one optical brightener, preferably in an amount of 0.01 to 2 wt.-%, more preferably 0.01 to 1 wt.-%; and

(d4) optionally at least one polymer, preferably in an amount of 0.01 to 7 wt.-%, more preferably 0.1 to 5 wt.-%; or

(e) is a fabric finisher and further comprises

(e1) at least one softening silicone, preferably an amino-functionalized silicone, preferably in an amount of 0.1 to 10 wt.-%, more preferably 0.1 to 2 wt.-%;

(e2) at least one perfume, preferably at least partially encapsulated in microcapsules, more preferably at least partially encapsulated in melamine-formaldehyde microcapsules, preferably in an amount of 0.01 to 3 wt.-%, more preferably 0.1 to 1 wt.-%;

(e3) optionally polyquaternium 10 in an amount of 0.1 to 20 wt.-%, preferably 0.1 to 13 wt.-%;

(e4) optionally polyquaternium 37 in an amount of 0.1 to 20 wt.-%, preferably 0.1 to 13 wt.-%;

(e5) optionally a plant-based esterquat, preferably a canola- or palm-based esterquat, in an amount of 0.1 to 20 wt.-%, preferably 0.1 to 13 wt.-%; and

(e6) optionally adipic acid, in an amount of 0.1 to 20 wt.-%, preferably 0.1 to 13 wt.-%; or

(f) is an acidic cleaning agent, preferably having a pH less than 6, and further comprises

(f1) plant-based or bio-based surfactants, preferably each in an amount of 0.1 to 5, more preferably each in an amount of 0.1 to 2 wt.-%;

(f2) at least one acidic biocide, preferably selected from acids, more preferably HCl and formic acid; and

(f3) at least one soil release, water repellant or water spreading polymer, preferably in an amount of 0.01 to 3 wt.-%, more preferably 0.01 to 0.5 wt.-%; or

(g) is a neutral cleaning agent, preferably having a pH between 6.0 and 7.5, and further comprises

(g1) plant-based or bio-based surfactants, preferably each in an amount of 0.1 to 5, more preferably each in an amount of 0.1 to 2 wt.-%;

(g2) at least one biocide, preferably selected from quaternary ammonium compounds and alcohols; and

(g3) at least one soil release, water repellant or water spreading polymer, preferably in an amount of 0.01 to 3 wt.-%, more preferably 0.01 to 0.5 wt.-%; or

(h) is an alkaline cleaning agent, preferably having a pH of more than 7.5, and further comprises

(h1) plant-based or bio-based surfactants, preferably each in an amount of 0.1 to 5, more preferably each in an amount of 0.1 to 2 wt.-%; or

(i) is a hand dishwashing agent, preferably liquid hand dishwashing agent, and further comprises

(i1) at least one anionic surfactant, preferably in an amount of 0.1 to 40 wt.-%, more preferably 5 to 30 wt.-%;

(i2) at least one amphoteric surfactant, preferably betain, preferably in an amount of 0.1 to 25 wt.-%, more preferably 1 to 15 wt.-%;

(i3) at least one nonionic surfactant, preferably in an amount of 0.1 to 25 wt.-%, more preferably 2 to 10 wt.-%;

(i4) at least one further enzyme, preferably selected from proteases, preferably in an amount of enzyme composition of up to 1 wt.-%, more preferably up to 0.6 wt.-%; or

(j) is an automatic dishwashing composition and further comprises

(j1) at least one builder selected from citrate, aminocarboxylates and combinations thereof, preferably in an amount of 5 to 30 wt.-%, more preferably 10 to 20 wt.-%;

(j2) at least one phosphonate builder, preferably in an amount of 0.1 to 5 wt.-%, more preferably 0.4 to 1.5 wt.-%;

(j3) at least one nonionic surfactant, preferably in an amount of 0.1 to 10 wt.-%, more preferably 1 to 5 wt.-%;

(j4) at least one bleaching system, comprising a bleaching agent, a bleach activator and a bleach catalyst, preferably in an amount of 0.1 to 50 wt.-%, more preferably 0.1 to 30 wt.-%; and

(j5) at least one polymer selected from sulfopolymers, cationic polymers and polyacrylates, preferably in an amount of 0.01 to 15 wt.-%, more preferably 2 to 10 wt.-%; or

(k) further comprises

(k1) at least one sulfopolymer, preferably in an amount of 1 to 15, more preferably 2 to 10 wt.-% and is preferably a dishwashing, more preferably an automatic dishwashing composition; or

(l) further comprises at least one adjunct ingredient selected from probiotics, preferably microbes, spores or combinations thereof; or

(m) is in unit dose form and comprises at least 2, preferably 2, 3, 4 or 5 separate compartments; or

(n) is a phosphate-free composition.

When in the following reference is made to “(cleaning) compositions of the invention” or “(cleaning) compositions as described herein” or “(cleaning) compositions as defined herein”, in various embodiments the above-specified compositions (a)-(n) are meant. However, the invention is not limited to those compositions and is intended to cover other suitable cleaning compositions that comprise the enzyme mixtures defined herein. When in the following reference is made to a “carbohydrase”, “amylase” or “mannanase” in the claimed combinations of a dispersin and a carbohydrase, the carbohydrase is one of the amylases/mannanases that are defined herein. Furthermore, if not indicated otherwise, all references to percentages in relation to the disclosed compositions relate to wt % relative to the total weight of the respective composition. It is understood that when reference is made to compositions that contain enzymes as defined herein, the respective composition contains at least one of each of the specified enzymes but can also comprise two or more of each enzyme type, such as two or more dispersins and/or two or more carbohydrases.

The invention further relates to the use of a cleaning composition comprising a dispersin, at least one carbohydrase and a cleaning component, wherein the carbohydrase is an amylase or a mannanase, as defined herein, for cleaning of an item, wherein the item is a textile or a surface as well as corresponding methods for cleaning of an item.

The invention further relates to methods of formulating a cleaning composition comprising a dispersin, at least one carbohydrase and a cleaning component, wherein the carbohydrase is an amylase or a mannanase, as defined herein, comprising adding a dispersin, a carbohydrase and at least one cleaning component. The invention further relates to a kit intended for deep cleaning, wherein the kit comprises a solution of an enzyme mixture comprising a dispersin and a carbohydrase, wherein the carbohydrase is an amylase or a mannanase.

The invention further relates to a method of deep cleaning of an item, comprising the steps of:
a) contacting the item with a cleaning composition comprising a dispersin, at least one carbohydrase and, optionally, a cleaning component, wherein the carbohydrase is an amylase or a mannanase; and
b) optionally rinsing the item, wherein the item is preferably a textile.

### DETAILED DESCRIPTION

Various enzymes are applied in cleaning processes each targeting specific types of soiling such as protein, starch and grease soiling. Enzymes are now standard ingredients in detergents for laundry and dish wash. The effectiveness of these commercial enzymes provides detergents which removes much of the soiling. However, organic stains such as biofilm, comprising polysaccharides (exopolysaccharides) and proteins, lipids and other organic substances may be sticky or gluing, which when present on textile, may give rise to redeposition or back-staining of soil resulting in a greying of the textile. Another potential drawback of organic stains is the malodor. Further, when dirty laundry items are washed together with less dirty laundry items the dirt present in the wash liquor tend to stick to organic stains as a result, hereof the laundry item is more “soiled” after wash than before wash. This effect may also be termed re-deposition. Rather than being simple stains, many organic stains comprise multiple different types of soils, such as those derived from biofilm and biofilm EPS (extracellular polymeric substances), body soiling such as sweat, skin cell debris, sebum, textile finishing such as ironing starch, and environmental soils such as dust and dirt. Some types of soil may also attract other types of soil, to form complex mixtures. Such complex stains are difficult to remove completely. It is not evident from the outset which enzymes or enzyme mixtures will provide increased or even synergistic removal of complex stains. Some enzymes may negatively impact other enzymes and some enzymes are not stable in detergents or is not stable under laundry conditions. The substances present in e.g. biofilm may be hidden or not accessible for the enzymes. Thus, enzyme blends are needed, which effectively remove such stains. The enzyme combinations of the present invention show effective removal of complex organic stains, involving but excluding biofilm staining or biofilm related staining. The dispersin component of the compositions of the invention provide effective removal of polysaccharide stains in particular poly-N-acetylglucosamine commonly abbreviated PNAG. Without being bound by theory it's assumed that the removal or reduction of the PNAG component of biofilm opens the structure making the stain more accessible to other stain removal enzymes such as carbohydrase. This combined action of the enzymes is surprising, and a result of the stains targeted by the compositions of the invention being particularly complex and difficult to remove using standard detergents. Commercial detergents currently available on the market do not target these complex stains. Such detergents target simple stains primarily composed of one component such as starch and protein. Exopolysaccharides are considered to play an important structural and functional role in the development and maintenance of microbial biofilm. Biofilm are often embedded within an extracellular polymeric substance (EPS). This matrix is a heterogeneous substance which may be composed of various substances such as extracellular DNA, proteins, lipids, and exopolysaccharides such as galactomannan and glycan. Exopolysaccharides are considered as core components of the extracellular matrix of most biofilm. The application of biofilm (component) reducing enzymes holds great potential for application in detergents and laundry either alone, or in particular in combination with conventional detergent enzymes such as the carbohydrases selected from mannanases and amylases.

Mannanases are mannan-degrading enzymes and includes α and β-mannanases, β-mannosidases and β-glucosidases. Amylases are glycoside hydrolases acting on α-1,4-glycosidic bonds and catalyzes the hydrolysis of starch. The combination of standard cleaning enzymes with an enzyme specifically targeting EPS polysaccharides such as PNAG is new. Though, standard detergent may be described as having enzymes such as amylases present, any specific combinations with the PNAG reducing enzymes dispersins is not suggested in the art.

The compositions of the invention comprise a blend of dispersin and a carbohydrase, wherein the carbohydrase is an amylase or a mannanase, as defined below. The compositions effectively reduce or remove organic components, such as starch and PNAG from surfaces such as textiles and hard surfaces e.g. dishes. Such components may be present in e.g. biofilm or biofilm EPS.

The compositions of the invention comprise a blend of dispersin and a carbohydrase, wherein the carbohydrase is an amylase or a mannanase, as defined herein below, and the compositions provides deep cleaning effect, when applied in e.g. laundry process.

The compositions of the invention comprise a blend of dispersin and a carbohydrase, wherein the carbohydrase is an amylase or a mannanase, as defined herein below, and the compositions effectively reduce or limit redeposition when applied in e.g. laundry process.

The compositions of the invention comprise a blend of dispersin and a carbohydrase, wherein the carbohydrase is an amylase or a mannanase, as defined herein below, and the compositions effectively reduce or limit malodor of e.g. textiles or hard surfaces such as dishes.

The compositions of the invention comprise a blend of dispersin and a carbohydrase, wherein the carbohydrase is an amylase or a mannanase, as defined herein below, and the compositions improve whiteness of textile.

A composition of the invention is a cleaning composition, the composition of the invention comprises at least one dispersin and at least one a carbohydrase, wherein the carbohydrase is an amylase or a mannanase, as defined herein below. Examples of useful dispersins are mentioned below in the sections “Polypeptides having dispersin activity”. The section “olypeptides having mannanase or amylase activity” defines the amylases and mannanases to be used in combination with the dispersins.

Polypeptides Having Hexosaminidase Activity (Hexosaminidases)

The term hexosaminidase includes “dispersin” and the abbreviation “Dsp”, and means a polypeptide having hexosaminidase activity, EC 3.2.1.- that catalyzes the hydrolysis of β-1,6-glycosidic linkages of N-acetyl-glucosamine polymers found e.g. in biofilm. The term hexosaminidase includes polypeptides having N-acetylglucosaminidase activity and β-N-acetylglucosaminidase activity. The term “polypeptide having hexosaminidase activity” may be used interchangeably with the term hexosaminidases and similarly the term “polypeptide having β-N-acetylglucosaminidase activity” may be used interchangeably with the term β-N-acetylglucosaminidases. For purposes of the present invention, hexosaminidase activity is determined according to the procedure described in Assay I. In a preferred embodiment, the polypeptide having hexosaminidase activity is a dispersin. In a preferred embodiment, the polypeptide having hexosaminidase activity is a β-N-acetylglucosaminidase targeting poly-β-1,6-N-acetylglucosamine.

In one embodiment, the invention relates to a composition comprising a carbohydrase, as defined herein, a hexosaminidase, preferably a β-N-acetylglucosaminidase e.g. a dispersin, and a cleaning component.

One embodiment of the invention relates to a composition comprising a hexosaminidase, preferably a β-N-acetylglucosaminidase e.g. a dispersin, polypeptide wherein the polypeptide is selected from the group consisting of polypeptides:
a) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 1,
b) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 2,
c) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 3,
d) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 4,
e) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 5,
f) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 6,
g) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 7,
h) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 8,
i) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 9,
j) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 10,
k) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 11,
l) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 12,
m) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 13,
n) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 14,
o) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 15,
p) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 16,
q) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 17,
r) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 18,
s) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 19,
t) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 20,
u) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 21,
v) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 22, and
w) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 23.

A polypeptide having hexosaminidase activity may be obtained from microorganisms of any genus. Preferably the hexosaminidase or the β-N-acetylglucosaminidase targeting poly-β-1,6-N-acetylglucosamine e.g. a dispersin, is obtained from Terribacillus, Curtobacterium, Aggregatibacter, Haemophilus, Actinobacillus, Lactobacillus or Staphylococcus, preferably Terribacillus or Lactobacillus. Alternatively, it may e.g. be obtained from Neisseria, Otariodibacter, Lactococcus, Frigoribacterium, Basfia, Weissella, Macrococcus or Leuconostoc.

In another embodiment, the polypeptide is an Aggregatibacter polypeptide, e.g., a polypeptide obtained from Aggregatibacter actinomycetemcomitans. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 1 and is obtained from Aggregatibacter, preferably Aggregatibacter actinomycetemcomitans.

In another embodiment, the polypeptide is a Haemophilus polypeptide, e.g., a polypeptide obtained from Haemophilus sputorum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2 and is obtained from Haemophilus, preferably Haemophilus sputorum.

In another embodiment, the polypeptide is an Actinobacillus polypeptide, e.g., a polypeptide obtained from Actinobacillus suis. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 3 and is obtained from Actinobacillus, preferably Actinobacillus suis.

In another embodiment, the polypeptide is an Actinobacillus polypeptide, e.g., a polypeptide obtained from Actinobacillus capsulatus DSM 19761. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 4 and is obtained from Actinobacillus, preferably Actinobacillus capsulatus DSM 19761.

In another embodiment, the polypeptide is an Actinobacillus polypeptide, e.g., a polypeptide obtained from Actinobacillus equuli subsp. equuli. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 5 and is obtained from Actinobacillus, preferably Actinobacillus equuli subsp. equuli.

In another embodiment, the polypeptide is an Aggregatibacter polypeptide, e.g., a polypeptide obtained from Aggregatibacter actinomycetemcomitans. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 6 and is obtained from Aggregatibacter, preferably Aggregatibacter actinomycetemcomitans.

In another embodiment, the polypeptide is an Aggregatibacter polypeptide, e.g., a polypeptide obtained from Aggregatibacter actinomycetemcomitans. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 7 and is obtained from Aggregatibacter, preferably Aggregatibacter actinomycetemcomitans.

In another embodiment, the polypeptide is an Actinobacillus polypeptide, e.g., a polypeptide obtained from Actinobacillus pleuropneumoniae. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 8 and is obtained from Actinobacillus, preferably Actinobacillus pleuropneumoniae.

In another embodiment, the polypeptide is a Curtobacterium polypeptide, e.g., a polypeptide obtained from Curtobacterium oceanosedimentum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 9 and is obtained from Curtobacterium, preferably Curtobacterium oceanosedimentum.

In another embodiment, the polypeptide is a Curtobacterium polypeptide, e.g., a polypeptide obtained from Curtobacterium flaccumfaciens. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 10 and is obtained from Curtobacterium, preferably Curtobacterium flaccumfaciens.

In another embodiment, the polypeptide is a Curtobacterium polypeptide, e.g., a polypeptide obtained from Curtobacterium luteum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 11 and is obtained from Curtobacterium, preferably Curtobacterium luteum.

In another embodiment, the polypeptide is a Curtobacterium polypeptide, e.g., a polypeptide obtained from Curtobacterium oceanosedimentum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 12 and is obtained from Curtobacterium, preferably Curtobacterium oceanosedimentum.

In another embodiment, the polypeptide is a Curtobacterium polypeptide, e.g., a polypeptide obtained from Curtobacterium leaf154. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 13 and is obtained from Curtobacterium, preferably Curtobacterium leaf154.

In another embodiment, the polypeptide having hexosaminidase activity is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 14 and is obtained from Terribacillus, preferably Terribacillus saccharophilus.

In another embodiment, the polypeptide is a Terribacillus polypeptide, e.g., a polypeptide obtained from Terribacillus goriensis. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 15 and is obtained from Terribacillus, preferably Terribacillus goriensis.

In another embodiment, the polypeptide is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 16 and is obtained from Terribacillus, preferably Terribacillus saccharophilus.

In another embodiment, the polypeptide is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 17 and is obtained from Terribacillus, preferably Terribacillus saccharophilus.

In another embodiment, the polypeptide is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 18 and is obtained from Terribacillus, preferably Terribacillus saccharophilus.

In another embodiment, the polypeptide is a Lactobacillus polypeptide, e.g., a polypeptide obtained from Lactobacillus paraplantarum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 19 and is obtained from Lactobacillus, preferably Lactobacillus paraplantarum.

In another embodiment, the polypeptide is a Lactobacillus polypeptide, e.g., a polypeptide obtained from Lactobacillus apinorum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 20 and is obtained from Lactobacillus, preferably Lactobacillus apinorum.

In another embodiment, the polypeptide is a Lactobacillus polypeptide, e.g., a polypeptide obtained from Lactobacillus paraplantarum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 21 and is obtained from Lactobacillus, preferably Lactobacillus paraplantarum.

In another embodiment, the polypeptide is a Staphylococcus polypeptide, e.g., a polypeptide obtained from Staphylococcus cohnii. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 22 and is obtained from Staphylococcus, preferably Staphylococcus cohnii.

In another embodiment, the polypeptide is a Staphylococcus polypeptide, e.g., a polypeptide obtained from Staphylococcus fleurettii. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 23 and is obtained from Staphylococcus, preferably Staphylococcus fleurettii.

The polypeptides useful in the present invention belong to the Glycoside Hydrolase family 20 (GH20, www.cazy.org). This family includes dispersins such as Dispersin B (DspB) which are β-N-acetylglucosaminidases belonging to the Glycoside Hydrolase 20 family.

The hexosaminidase can be included in the cleaning composition of the present invention at a level of from 0.01 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, from 250 ppm to 500 ppm. All enzyme concentrations given herein are, if not indicated otherwise, meant to refer to the active protein and not the enzyme formulation.

The hexosaminidase can be included in the wash liquor solution at a level of from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, from 0.1 ppm to 10 ppm, from 0.2 ppm to 10 ppm, from 0.5 ppm to 5 ppm.

The dispersins above may be combined with any of the carbohydrases below to form a blend to be added to a composition according to the invention. In various embodiments, the dispersin is a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 17.

Polypeptides Having Carbohydrase Activity (Carbohydrases)

Carbohydrase is a protein/enzyme that catalyse carbohydrates to break down carbohydrates to e.g. simple sugar such as monosaccharides. Thus, carbohydrases are any of a group of enzymes that promote hydrolysis of a carbohydrate. Starch hydrolyzing carbohydrases (e.g. amylases) work on e.g. amylose and amylopectin and non-starch carbohydrases includes enzymes which hydrolyze polymers made up of carbon sugars e.g. cellulases which will ultimately produce glucose when complete hydrolysis is achieved. Another example is lactase which hydrolyses lactose to glucose and galactose. Examples of carbohydrases include amylases, cellulases, xyloglucanases and mannanases. The carbohydrases to be incorporated in a composition according to the invention are selected from mannanases and amylases as described in the following

Polypeptides Having Mannanase Activity

The term “mannanase” is defined here as an enzyme that hydrolyses compounds known as mannans. Mannanases are enzyme catalyzing hydrolyses of 1,4-beta-D-mannosidic linkages in mannans, galactomannans, glucomannans, and galactoglucomannans. Mannans are polysaccharides with a backbone of β-1,4-linked D-mannopyranosyl residues, which can contain galactose or acetyl substitutions and may have glucose residues in the backbone. The main enzyme type participating in the degradation of mannans are endo-1,4-β-mannanases (EC 3.2.1.78), which hydrolyze the internal glycoside bonds in the mannan backbone. The term “mannanase activity” is defined as an enzyme catalyzed hydrolysis of mannan, for purposes of the present invention, mannanase activity is determined according to the procedure described in the Assay II. The present invention provides a cleaning composition comprising a dispersin and a mannanase enzyme comprising a polypeptide having mannan endo-1,4-beta-mannosidase activity (EC 3.2.1.78) that catalyzes the hydrolysis of 1,4-beta-D-mannosidic linkages in mannans, galactomannans and/or glucomannans.

In the following sequences, insertions are described by using the one letter code “Z” on the left side of the position number and including a number (e.g., 0.01) before each amino acid being inserted therein to indicate the order of insertion. For example, the insertion of one amino acid, glutamine (Q), at position 298 would be depicted as “Z298.01Q”. For a deletion, the one letter code “Z” is on the right side of the position number. For example, the deletion of an alanine (A) from position 100 would be depicted as A100Z.

The term “variant”, as used herein, relates to a variant that has a specified degree of sequence homology/identity with a parent sequence. For example, a variant may have at least 59, 60, 65, 70, 75, 80, 81, 82, 83, 84, 85, 86, 87, 88, 89, 90, 91, 92, 93, 94, 95, 96, 97, 98, or 99% sequence identity with a reference (parent) sequence. Sequence identity may be determined using known programs, such as BLAST, ALIGN, and CLUSTAL using standard parameters. Software for performing BLAST analyses is publicly available through the National Center for Biotechnology Information (NCBI). The term “mannanase variant” refers to a polypeptide that is derived from a reference polypeptide by the substitution, addition, or deletion, of one or more amino acids, typically by recombinant DNA techniques. A mannanase variant may differ from a reference polypeptide by a small number of amino acid residues and may be defined by the level of primary amino acid sequence homology/identity with the reference polypeptide over the length of the catalytic domain. For example, a mannanase variant has at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity with a reference polypeptide. The reference polypeptide includes naturally occurring and recombinant mannanases within the GH5 8 sub family of mannanases (endo-1,4 β-mannosidases, EC 3.2.1.78).

According to the invention, the mannanases useful in the present invention are selected from the group consisting of:

A mannanase variant, or a recombinant polypeptide or an active fragment thereof comprising an amino acid sequence comprising two or more modifications selected from:
(1) (a) one or more substitutions at one or more positions selected from 1, 2, 3, 4, 6, 10, 19, 28, 30, 38, 59, 60, 61, 62, 63, 66, 67, 68, 70, 71, 74, 75, 78, 80, 82, 93, 97, 103, 111, 124, 129, 131, 135, 136, 139, 143, 150, 167, 168, 184, 213, 214, 217, 225, 228, 235, 242, 244, 258, 259, 261, 283, and 284, and (b) an insertion at position 298; or
(2) one or more substitutions at one or more positions selected from 19, 38, 59, 67, 68, 71, 74, 97, 129, 167, 168, 184, 225, 228, 235, 242, 244, 258, and 261;

wherein the amino acid positions of the variant or recombinant polypeptide or active fragment thereof are numbered by correspondence with the amino acid sequence of SEQ ID NO: 24.

In various embodiments, said variant or recombinant polypeptide or active fragment thereof comprises two or more modifications selected from:
i) one or more substitutions at one or more positions selected from M1X, A2X, T3X, G4X, Y6X, N10X, P19X, G28X, S30X, T38X, S59X, L60X, Y61X, T62X, K63X, L66X, N67X, A68X, K70X, N71X, N74X, V75X, Q78X, K80X, 182X, K93X, N97X, V103X, E111X, 1124X, Y129X, T131X, S135X, A136X, D139X, K143X, N150X, F167X, P168X, Q184X, N213X, K214X, A217X, G225X, T228X, Y235X, Q242X, K244X, S258X, G259X, N261X, D283X, and T284X, and an insertion at position Z298.01X; wherein X is any amino acid; or
ii) one or more substitutions at one or more positions selected from P19X, T38X, S59X, N67X, A68X, N71X, N74X, N97X, Y129X, F167X, P168X, Q184X, G225X T228X, Y235X, Q242X, K244X, S258X, and N261X, wherein X is any amino acid;
wherein the amino acid positions of the variant or recombinant polypeptide or active fragment thereof are numbered by correspondence with the amino acid sequence of SEQ ID NO: 24.

In various embodiments of said mannanase variant, or a recombinant polypeptide or active fragment thereof, said variant or recombinant polypeptide or active fragment thereof comprises two or more modifications selected from:
i) one or more substitutions at one or more positions selected from X1V, X1 L, X2S, X3R, X4S, X6S, X6E, X10T, X10S, X19E, X28A, X28S, X30T, X38E, X59D, X59V, X60Q, X61W, X62E, X63R, X63L, X66V, X67D, X68S, X70R, X71D, X74E, X74S, X75L, X78D, X78H, X80T, X82M, X93R, X97D, X97L, X103I, X111D, X111S, X124V, X129M, X131A, X135L, X136L, X139M, X143Q, X143R, X150T, X167Y, X168A, X168S, X184D, X184L, X213A, X214I, X217P, X225C, X225P, X228V, X235L, X242L, X244L, X258D, X259P, X261Q, X261R, X283S, X284A, and X284E, and an insertion at position Z298.01Q; wherein X is any amino acid; or
ii) one or more substitutions at one or more positions selected from X19E, X38E, X59V, X67D, X68S, X71D, X74E, X74S, X97D, X97L, X129M, X167Y, X168A, X168S, X184D, X184L, X225C, X225P, X228V, X235L, X242L, X244L, X258D, X261Q, and X261R, wherein X is any amino acid;

wherein the amino acid positions of the variant or recombinant polypeptide or active fragment thereof are numbered by correspondence with the amino acid sequence of SEQ ID NO: 24.

In various embodiments, the mannanase variant, or a recombinant polypeptide or active fragment thereof comprises an amino acid sequence comprising two or more modifications selected from:
i) one or more substitutions at one or more positions selected from M1V, M1L, A2S, T3R, G4S, Y6S, Y6E, N10T, N10S, P19E, G28A, G28S, S30T, T38E, S59D, S59V, L60Q, Y61W, T62E, K63R, K63L, L66V, N67D, A68S, K70R, N71D, N74E, N74S, V75L, Q78D, Q78H, K80T, 182M, K93R, N97D, N97L, V103I, E111D, Ell1S, I124V, Y129M, T131A, T135L, A136L, D139M, K143Q, K143R, N150T, F167Y, P168A, P168S, Q184D, Q184L, N213A, K214I, A217P, G225C, G225P, T228V, Y235L, Q242L, K244L, S258D, G259P, N261Q, N261R, D283S, T284A, and T284E, and an insertion at position Z298.01Q; or
ii) one or more substitutions at one or more positions selected from P19E, T38E, S59D, S59V, N67D, A68S, N71D, N74E, N74S, N97D, N97L, Y129M, F167Y, P168A, P168S, Q184D, Q184L, G225C, G225P, T228V, Y235L, Q242L, K244L, S258D, N261Q, and N261R;

wherein the amino acid positions of the variant or recombinant polypeptide or active fragment thereof are numbered by correspondence with the amino acid sequence of SEQ ID NO: 24.

In various embodiments, the two or more modifications are selected from:
i) one or more substitutions at one or more positions selected from the following 129 and 244; 129 and 143 and 244; 38 and 258; 38 and 143 and 258; 19 and 184; 19 and 143 and 184; 97 and 225; 97 and 143 and 225; 60 and 61; 67 and 168; 67 and 143 and 168; 63 and 71; 63 and 71 and 143; 228 and 235; and 143 and 228 and 235;
ii) one or more substitutions at one or more positions selected from Y129X and K244X; Y129X and K143X and K244X; T38X and S258X; T38X and K143X and S258X; P19X and Q184X; P19X and K143X and Q184X; N97X and G225X; N97X and K143X and G225X; L60X and Y61X; N67X and P168X; N67X and K143X and P168X; K63X and N71X; K63X and N71X and K143X; T228X and Y235X; K143X and T228X and Y235X; wherein X is any amino acid;
iii) one or more substitutions at one or more positions selected from X129M and X244L; X129M and X143Q and X244L; X38E and X258D; X38E and X143Q and X258D; X19E and X184D; X19E and X143Q and X184D; X19E and X184L; X19E and X143Q and X184L; X97D and X225C; X97D and X143Q and X225C; X97D and X225P; X97D and X143Q and X225P; X60Q and X61W; X67D and X168S; X67D and X143Q and X168S; X63L and X71 D; X63L and X71D and X143Q; X63R and X71D; X63R and X71D and X143Q; X228V and X235L; X143Q and X228V and X235L; wherein X is any amino acid; and
iv) Y129M and K244L; Y129M and K143Q and K244L; T38E and S258D; T38E and K143Q and S258D; P19E and Q184D; P19E and K143Q and Q184D; P19E and Q184L; P19E and K143Q and Q184L; N97D and G225C; N97D and K143Q and G225C; L60Q and Y61W; N97D and G225P; N97D and K143Q and G225P; N67D and P168S; N67D and K143Q and P168S; K63L and N71D; K63L and N71D and K143Q; K63R and N71D; K63R and N71D and K143Q; T228V and Y235L; K143Q and T228V and Y235L;

wherein the amino acid positions of the variant or recombinant polypeptide or active fragment thereof are numbered by correspondence with the amino acid sequence of SEQ ID NO: 24.

In various embodiments, said two or more modifications are selected from P19E and T38E and K63L and N71D and Y129M and Q184L and K244L and S258D and N261R; N67D and Y129M and P168S and Q184L and K244L and S258D and G259P; P19E and K63L and N67D and Q78D and K80T and N97D and Y129M and G225C and T228V and K244L; P19E and T38E and N67D and N97D and Y129M and P168 S and Q184L and K244L and S258D and N261R; P19E and T38E and N67D and N71D and Q78D and K80T and N97D and Y129M and P168S and G225C and K244L and S258D and N261R; T38E and K63L and N71D and N97D and Y129M and Q184L and G225C and T228V and Q242L and K244L and S258D and N261R; P19E and K63L and N71D and N97D and Y129M and Q184L and G225C and K244L and S258D and G259P; N10T and T38E and S59V and L60Q and K63R and L66V and A68S and N74S and V75L and N97D and V103I and Y129M and F167Y and Q184L and A217P and G225C and Y235L and K244L and S258D and N261R and Z298.01Q; P19E and T38E and N67D and N71 D and N97D and Y129M and F167Y and Q184L and A217P and K244L and S258D and N261R; T38E and K63L and N67D and Q78D and K80T and N97D and Y129M and P168S and Q184L and K244L and S258D and N261R; P19E and T38E and N67D and Y129M and P168S and Q184L and K244L and S258D and N261R; P19E and N67D and N97D and Y1129M and P168S and Q184L and K244L; P19E and T38E and K63L and N71D and Y129M and P168S and G225C and T228V and K244L and S258D and N261R; P19E and T38E and N67D and N97D and Q184L and A217P and G225C and T228V and Y235L and K244L and S258D and N261R; N10T and P19E and G28S and S30T and T38E and N67D and N71D and N97D and Y129M and P168S and Q184L and G225C and Y235L and K244L and S258D and N261R and Z298.01Q; P19E and T38E and S59V and L60Q and K63R and N67D and N97D and V103I and Y129M and K143Q and F167Y and Q184L and G225C and Y235L and K244L and S258D and N261R and Z298.01Q; P19E and T38E and N67D and N71D and Q78D and K80T and N97D and Y129M and P168S and G225C and T228V and K244L and S258D and N261R and Z298.01Q; P19E and T38E and S59V and L60Q and K63L and K70R and N71D and Q78D and K80T and N97D and E111D and Y129M and Q184L and G225C and T228V and Y235L and K244L and S258D and N261R and Z298.01Q; N10T and T38E and K63L and N71D and N97D and V103I and Y129M and F167Y and Q184L and G225C and T228V and Y235L and K244L and S258D and N261R and Z298.01Q; N10T and P19E and T38E and N67D and Q78D and K80T and N97D and Y129M and K143Q and Q184L and A217P and G225C and T228V and Y235L and K244L and S258D and N261R and Z298.01Q; N10T and P19E and T38E and S59V and L60Q and K63L and N97D and V103I and Y129M and F167Y and Q184L and G225C and Y235L and K244L and S258D and N261R and Z298.01Q; P19E and S30T and T38E and S59V and L60Q and K63R and N67D and N97D and V103I and Y129M and F167Y and Q184L and G225C and T228V and Y235L and K244L and S258D and N261R and Z298.01Q; P19E and S30T and T38E and S59V and L60Q and K63R and N67D and Q78D and K80T and N97D and I124V and Y129M and K143Q and F167Y and Q184L and G225C and Y235L and K244L and S258D and N261R and Z298.01Q; N10S and P19E and S30T and T38E and S59V and L60Q and K63L and N67D and Q78H and K80T and 182M and N97D and Y129M and K143Q and F167Y and Q184L and G225C and Y235L and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; G4S and N10T and P19E and T38E and N67D and Q78D and K80T and N97D and Y129M and Q184L and G225C and T228V and Y235L and K244L and S258D and N261R and Z298.01Q; N10T and P19E and S30T and T38E and S59V and L60Q and K63L and K70R and N71 D and Q78D and K80T and N97D and Y129M and T131A and F167Y and Q184L and G225C and Y235L and K244L and S258D and N261R and Z298.01Q; N10T and P19E and S30T and T38E and S59V and L60Q and K63L and K70R and N71D and Q78D and K80T and N97D and E111D and Y129M and P168S and Q184L and G225C and T228V and Y235L and K244L and S258D and N261R and Z298.01Q; P19E and S30T and T38E and S59V and L60Q and K63R and N67D and N97D and Y129M and P168S and Q184L and K214I and G225C and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; M1V and P19E and S30T and T38E and T62E and N67D and N71D and Q78D and N97D and Y129M and K143R and F167Y and P168S and Q184L and G225C and Y235L and K244L and S258D and N261R and T284A and Z298.01Q; Y6E and N10T and P19E and G28S and S30T and T38E and K63L and N67D and N71D and N97D and E111S and Y129M and S135L and P168S and Q184L and G225C and T228V and Y235L and K244L and S258D and N261Q and D283S and Z298.01Q; N10T and P19E and S30T and T38E and S59V and L60Q and K63R and N67D and N71D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; M1L and N10T and P19E and G28A and S30T and T38E and K63L and N67D and N71D and Q78D and N97D and Y129M and A136L and P168A and Q184L and N213A and G225C and Y235L and K244L and S258D and N261R and Z298.01Q; P19E and T38E and S59V and K63R and N67D and N97D and V103I and Y129M and F167Y and Q184L and G225C and T228V and Y235L and K244L and S258D and N261R and Z298.01Q; N10 and P19E and G28A and S30T and T38E and K63R and N67D and N97D and Y129M and Q184L and G225C and T228V and Y235L and K244L and S258D and N261R and Z298.01Q; T3R and N10 and P19E and G28A and S30T and T38E and T62E and N67D and N71D and K93R and N97L and E111S and Y129M and D139M and P168S and Q184L and G225C and Y235L and K244L and S258D and N261Q and Z298.01Q; N10T and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; P19E and K63L and N71D and Y129M and P168S and Q184L and G225C and K244L; P19E and N67D and N71D and Q78D and K80T and N97D and Y129M and P168S and Q184L and K244L; P19E and T38E and N67D and Y129M and P168S and Q184L and T228V and K244L; P19E and T38E and N67D and Y129M and Q184L and K244L and S258D and N261R; P19E and K63L and N71D and Y129M and P168S and Q184L and K244L and S258D and N261R; P19E and T38E and K63L and N71 D and Y129M and P168S and Q184L and K244L and S258D and G259P; K63L and N71D and Y129M and K143R and P168S and Q184L and G225C and T228V and K244L and S258D and G259P; or P19E and T38E and K63L and N71D and Y129M and P168S and Q184L and K244L and S258D and N261R, wherein the amino acid positions of the variant or recombinant polypeptide or active fragment thereof are numbered by correspondence with the amino acid sequence of SEQ ID NO: 24.

In various embodiments, said two or more modifications are selected from P19E and T38E and N67D and N97D and Y129M and P168S and Q184L and K244L and S258D and N261R; N10T and P19E and G28S and S30T and T38E and N67D and N71D and N97D and Y129M and P168S and Q184L and G225C and Y235L and K244L and S258D and N261R and Z298.01Q; P19E and S30T and T38E and S59V and L60Q and K63R and N67D and N97D and V103I and Y129M and F167Y and Q184L and G225C and T228V and Y235L and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; N10T and P19E and S30T and T38E and S59V and L60Q and K63R and N67D and N71D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; T3R and N10T and P19E and G28A and S30T and T38E and T62E and N67D and N71D and K93R and N97L and E111S and Y129M and D139M and P168S and Q184L and G225C and Y235L and K244L and S258D and N261Q and Z298.01Q; N10T and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; wherein the amino acid positions of the variant or recombinant polypeptide or active fragment thereof are numbered by correspondence with the amino acid sequence of SEQ ID NO: 24.

In various embodiments, said two or more modifications are selected from N67D and Y129M and P168S and Q184L and K244L and S258D and G259P; P19E and T38E and N67D and N97D and Y129M and P168S and Q184L and K244L and S258D and N261R; P19E and T38E and N67D and N71D and Q78D and K80T and N97D and Y129M and P168S and G225C and K244L and S258D and N261R; T38E and K63L and N67D and Q78D and K80T and N97D and Y129M and P168S and Q184L and K244L and S258D and N261R; P19E and T38E and N67D and Y129M and P168S and Q184L and K244L and S258D and N261R; P19E and N67D and N97D and Y129M and P168 S and Q184L and K244L; N10 and P19E and G28S and S30T and T38E and N67D and N71D and N97D and Y129M and P168S and Q184L and G225C and Y235L and K244L and S258D and N261R and Z298.01Q; P19E and T38E and N67D and N71D and Q78D and K80T and N97D and Y129M and P168S and G225C and T228V and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; P19E and S30T and T38E and S59V and L60Q and K63R and N67D and N97D and Y129M and P168S and Q184L and K214I and G225C and Y235L and K244L and S258D and N261R and Z298.01Q; N10 and P19E and S30T and T38E and S59V and L60Q and K63R and N67D and N97D and Y129M and K143Q and P168S and Q184L and G225P and T228V and Y235L and K244L and S258D and N261R and Z298.01Q; M1V and P19E and S30T and T38E and T62E and N67D and N71D and Q78D and N97D and Y129M and K143R and F167Y and P168S and Q184L and G225C and Y235L and K244L and S258D and N261R and T284A and Z298.01Q; Y6E and N10T and P19E and G28 S and S30T and T38E and K63L and N67D and N71D and N97D and E111 S and Y129M and S135L and P168S and Q184L and G225C and T228V and Y235L and K244L and S258D and N261Q and D283S and Z298.01Q; N10 and P19E and S30T and T38E and S59V and L60Q and K63R and N67D and N71D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; T3R and N10T and P19E and G28A and S30T and T38E and T62E and N67D and N71D and K93R and N97L and E111S and Y129M and D139M and P168S and Q184L and G225C and Y235L and K244L and S258D and N261Q and Z298.01Q; N10T and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; P19E and N67D and N71D and Q78D and K80T and N97D and Y129M and P168S and Q184L and K244L; P19E and T38E and N67D and Y129M and P168S and Q184L and T228V and K244L; wherein the amino acid positions of the variant or recombinant polypeptide or active fragment thereof are numbered by correspondence with the amino acid sequence of SEQ ID NO: 24.

In various embodiments, said two or more modifications are selected from P19E and T38E and K63L and N71D and Y129M and Q184L and K244L and S258D and N261R; N67D and Y129M and P168S and Q184L and K244L and S258D and G259P; P19E and K63L and N67D and Q78D and K80T and N97D and Y129M and G225C and T228V and K244L; P19E and T38E and N67D and N97D and Y129M and P168S and Q184L and K244L and S258D and N261R; P19E and T38E and N67D and N71D and Q78D and K80T and N97D and Y129M and P168S and G225C and K244L and S258D and N261R; T38E and K63L and N71D and N97D and Y129M and Q184L and G225C and T228V and Q242L and K244L and S258D and N261R; P19E and K63L and N71D and N97D and Y129M and Q184L and G225C and K244L and S258D and G259P; N10T and T38E and S59V and L60Q and K63R and L66V and A68S and N74S and V75L and N97D and V103I and Y129M and F167Y and Q184L and A217P and G225C and Y235L and K244L and S258D and N261R and Z298.01Q; P19E and T38E and N67D and N71 D and N97D and Y129M and F167Y and Q184L and A217P and K244L and S258D and N261R; T38E and K63L and N67D and Q78D and K80T and N97D and Y129M and P168S and Q184L and K244L and S258D and N261R; P19E and T38E and N67D and Y129M and P168 S and Q184L and K244L and S258D and N261R; P19E and N67D and N97D and Y129M and P168 S and Q184L and K244L; P19E and T38E and K63L and N71D and Y129M and P168S and G225C and T228V and K244L and S258D and N261R; N10T and P19E and G28S and S30T and T38E and N67D and N71D and N97D and Y129M and P168S and Q184L and G225C and Y235L and K244L and S258D and N261R and Z298.01Q; P19E and T38E and S59V and L60Q and K63R and N67D and N97D and V103I and Y129M and K143Q and F167Y and Q184L and G225C and Y235L and K244L and S258D and N261R and Z298.01Q; P19E and T38E and N67D and N71D and Q78D and K80T and N97D and Y129M and P168S and G225C and T228V and K244L and S258D and N261R and Z298.01Q; P19E and T38E and S59V and L60Q and K63L and K70R and N71 D and Q78D and K80T and N97D and E111D and Y129M and Q184L and G225C and T228V and Y235L and K244L and S258D and N261R and Z298.01Q; N10 and T38E and K63L and N71D and N97D and V103I and Y129M and F167Y and Q184L and G225C and T228V and Y235L and K244L and S258D and N261R and Z298.01Q; N10 and P19E and T38E and N67D and Q78D and K80T and N97D and Y129M and K143Q and Q184L and A217P and G225C and T228V and Y235L and K244L and S258D and N261R and Z298.01Q; N10T and P19E and T38E and S59V and L60Q and K63L and N97D and V103I and Y129M and F167Y and Q184L and G225C and Y235L and K244L and S258D and N261R and Z298.01Q; P19E and S30T and T38E and S59V and L60Q and K63R and N67D and N97D and V103I and Y129M and F167Y and Q184L and G225C and T228V and Y235L and K244L and S258D and N261R and Z298.01Q; P19E and S30T and T38E and S59V and L60Q and K63R and N67D and Q78D and K80T and N97D and I124V and Y129M and K143Q and F167Y and Q184L and G225C and Y235L and K244L and S258D and N261R and Z298.01Q; N10S and P19E and S30T and T38E and S59V and L60Q and K63L and N67D and Q78H and K80T and 182M and N97D and Y129M and K143Q and F167Y and Q184L and G225C and Y235L and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; G4S and N10T and P19E and T38E and N67D and Q78D and K80T and N97D and Y129M and Q184L and G225C and T228V and Y235L and K244L and S258D and N261R and Z298.01Q; N10T and P19E and S30T and T38E and S59V and L60Q and K63L and K70R and N71D and Q78D and K80T and N97D and Y129M and T131A and F167Y and Q184L and G225C and Y235L and K244L and S258D and N261R and Z298.01Q; N10T and P19E and S30T and T38E and S59V and L60Q and K63L and K70R and N71D and Q78D and K80T and N97D and E111D and Y129M and P168S and Q184L and G225C and T228V and Y235L and K244L and S258D and N261R and Z298.01Q; P19E and S30T and T38E and S59V and L60Q and K63R and N67D and N97D and Y129M and P168S and Q184L and K214I and G225C and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; M1V and P19E and S30T and T38E and T62E and N67D and N71D and Q78D and N97D and Y129M and K143R and F167Y and P168S and Q184L and G225C and Y235L and K244L and S258D and N261R and T284A and Z298.01Q; Y6E and N10T and P19E and G28S and S30T and T38E and K63L and N67D and N71D and N97D and E111 S and Y129M and S135L and P168S and Q184L and G225C and T228V and Y235L and K244L and S258D and N261Q and D283S and Z298.01Q; N10T and P19E and S30T and T38E and S59V and L60Q and K63R and N67D and N71D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; M1 L and N10T and P19E and G28A and S30T and T38E and K63L and N67D and N71D and Q78D and N97D and Y129M and A136L and P168A and Q184L and N213A and G225C and Y235L and K244L and S258D and N261R and Z298.01Q; P19E and T38E and S59V and K63R and N67D and N97D and V103I and Y129M and F167Y and Q184L and G225C and T228V and Y235L and K244L and S258D and N261R and Z298.01Q; N10T and P19E and G28A and S30T and T38E and K63R and N67D and N97D and Y129M and Q184L and G225C and T228V and Y235L and K244L and S258D and N261R and Z298.01Q; T3R and N10T and P19E and G28A and S30T and T38E and T62E and N67D and N71 D and K93R and N97L and E111S and Y129M and D139M and P168S and Q184L and G225C and Y235L and K244L and S258D and N261Q and Z298.01Q; N10T and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; P19E and K63L and N71D and Y129M and P168S and Q184L and G225C and K244L; P19E and N67D and N71D and Q78D and K80T and N97D and Y129M and P168 S and Q184L and K244L; P19E and T38E and N67D and Y129M and P168S and Q184L and T228V and K244L; P19E and T38E and N67D and Y129M and Q184L and K244L and S258D and N261R; P19E and K63L and N71D and Y129M and P168S and Q184L and K244L and S258D and N261R; P19E and T38E and K63L and N71 D and Y129M and P168S and Q184L and K244L and S258D and G259P; K63L and N71D and Y129M and K143R and P168S and Q184L and G225C and T228V and K244L and S258D and G259P; and P19E and T38E and K63L and N71D and Y129M and P168S and Q184L and K244L and S258D and N261R; wherein the amino acid positions of the variant or recombinant polypeptide or active fragment thereof are numbered by correspondence with the amino acid sequence of SEQ ID NO: 24.

In various embodiments, said two or more modifications are selected from P19E and T38E and S59V and L60Q and K63R and N67D and N97D and V103I and Y129M and K143Q and F167Y and Q184L and G225C and Y235L and K244L and S258D and N261R and Z298.01Q; N10 and P19E and T38E and N67D and Q78D and K80T and N97D and Y129M and K143Q and Q184L and A217P and G225C and T228V and Y235L and K244L and S258D and N261R and Z298.01Q; P19E and S30T and T38E and S59V and L60Q and K63R and N67D and Q78D and K80T and N97D and I124V and Y129M and K143Q and F167Y and Q184L and G225C and Y235L and K244L and S258D and N261R and Z298.01Q; N10S and P19E and S30T and T38E and S59V and L60Q and K63L and N67D and Q78H and K80T and 182M and N97D and Y129M and K143Q and F167Y and Q184L and G225C and Y235L and K244L and S258D and N261R and Z298.01Q; N10 and P19E and S30T and T38E and S59V and L60Q and K63R and N67D and N97D and Y129M and K143Q and P168S and Q184L and G225C and T228V and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; N10 and P19E and S30T and T38E and S59V and L60Q and K63R and N67D and N71D and N97D and V103I and Y129M and K143Q and P168S and Q184L and G225P and T228V and Y235L and K244L and S258D and N261R and Z298.01Q; and N10 and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; wherein the amino acid positions of the variant or recombinant polypeptide or active fragment thereof are numbered by correspondence with the amino acid sequence of SEQ ID NO: 24.

In various embodiments, said two or more modifications are selected from P19E and T38E and K63L and N71D and Y129M and Q184L and K244L and S258D and N261R; P19E and T38E and N67D and N97D and Y129M and P168S and Q184L and K244L and S258D and N261R; P19E and T38E and N67D and N71D and Q78D and K80T and N97D and Y129M and P168S and G225C and K244L and S258D and N261R; T38E and K63L and N71D and N97D and Y129M and Q184L and G225C and T228V and Q242L and K244L and S258D and N261R; N10T and T38E and S59V and L60Q and K63R and L66V and A68S and N74S and V75L and N97D and V103I and Y129M and F167Y and Q184L and A217P and G225C and Y235L and K244L and S258D and N261R and Z298.01Q; P19E and T38E and N67D and N71D and N97D and Y129M and F167Y and Q184L and A217P and K244L and S258D and N261R; T38E and K63L and N67D and Q78D and K80T and N97D and Y129M and P168S and Q184L and K244L and S258D and N261R; P19E and T38E and N67D and Y129M and P168S and Q184L and K244L and S258D and N261R; P19E and T38E and K63L and N71D and Y129M and P168S and G225C and T228V and K244L and S258D and N261R; P19E and T38E and N67D and N97D and Q184L and A217P and G225C and T228V and Y235L and K244L and S258D and N261R; N10T and P19E and G28S and S30T and T38E and N67D and N71D and N97D and Y129M and P168S and Q184L and G225C and Y235L and K244L and S258D and N261R and Z298.01Q; P19E and T38E and S59V and L60Q and K63R and N67D and N97D and V103I and Y129M and K143Q and F167Y and Q184L and G225C and Y235L and K244L and S258D and N261R and Z298.01Q; P19E and T38E and N67D and N71D and Q78D and K80T and N97D and Y129M and P168S and G225C and T228V and K244L and S258D and N261R and Z298.01Q; P19E and T38E and S59V and L60Q and K63L and K70R and N71D and Q78D and K80T and N97D and E111D and Y129M and Q184L and G225C and T228V and Y235L and K244L and S258D and N261R and Z298.01Q; N10T and T38E and K63L and N71D and N97D and V103I and Y129M and F167Y and Q184L and G225C and T228V and Y235L and K244L and S258D and N261R and Z298.01Q; N10T and P19E and T38E and N67D and Q78D and K80T and N97D and Y129M and K143Q and Q184L and A217P and G225C and T228V and Y235L and K244L and S258D and N261R and Z298.01Q; N10T and P19E and T38E and S59V and L60Q and K63L and N97D and V103I and Y129M and F167Y and Q184L and G225C and Y235L and K244L and S258D and N261R and Z298.01Q; P19E and S30T and T38E and S59V and L60Q and K63R and N67D and N97D and V103I and Y129M and F167Y and Q184L and G225C and T228V and Y235L and K244L and S258D and N261R and Z298.01Q; P19E and S30T and T38E and S59V and L60Q and K63R and N67D and Q78D and K80T and N97D and I124V and Y129M and K143Q and F167Y and Q184L and G225C and Y235L and K244L and S258D and N261R and Z298.01Q; N10S and P19E and S30T and T38E and S59V and L60Q and K63L and N67D and Q78H and K80T and 182M and N97D and Y129M and K143Q and F167Y and Q184L and G225C and Y235L and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; G4S and N10T and P19E and T38E and N67D and Q78D and K80T and N97D and Y129M and Q184L and G225C and T228V and Y235L and K244L and S258D and N261R and Z298.01Q; N10T and P19E and S30T and T38E and S59V and L60Q and K63L and K70R and N71D and Q78D and K80T and N97D and Y129M and T131A and F167Y and Q184L and G225C and Y235L and K244L and S258D and N261R and Z298.01Q; N10T and P19E and S30T and T38E and S59V and L60Q and K63L and K70R and N71D and Q78D and K80T and N97D and E111D and Y129M and P168S and Q184L and G225C and T228V and Y235L and K244L and S258D and N261R and Z298.01Q; P19E and S30T and T38E and S59V and L60Q and K63R and N67D and N97D and Y129M and P168S and Q184L and K214I and G225C and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; M1V and P19E and S30T and T38E and T62E and N67D and N71D and Q78D and N97D and Y129M and K143R and F167Y and P168S and Q184L and G225C and Y235L and K244L and S258D and N261R and T284A and Z298.01Q; Y6E and N10 and P19E and G28S and S30T and T38E and K63L and N67D and N71D and N97D and E111S and Y129M and S135L and P168S and Q184L and G225C and T228V and Y235L and K244L and S258D and N261Q and D283S and Z298.01Q; N10T and P19E and S30T and T38E and S59V and L60Q and K63R and N67D and N71 D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; M1 L and N10 and P19E and G28A and S30T and T38E and K63L and N67D and N71D and Q78D and N97D and Y129M and A136L and P168A and Q184L and N213A and G225C and Y235L and K244L and S258D and N261R and Z298.01Q; P19E and T38E and S59V and K63R and N67D and N97D and V103I and Y129M and F167Y and Q184L and G225C and T228V and Y235L and K244L and S258D and N261R and Z298.01; N10T and P19E and G28A and S30T and T38E and K63R and N67D and N97D and Y129M and Q184L and G225C and T228V and Y235L and K244L and S258D and N261R and Z298.01Q; T3R and N10T and P19E and G28A and S30T and T38E and T62E and N67D and N71 D and K93R and N97L and E111 S and Y129M and D139M and P168S and Q184L and G225C and Y235L and K244L and S258D and N261Q and Z298.01Q; N10T and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; P19E and T38E and N67D and Y129M and Q184L and K244L and S258D and N261R; P19E and T38E and K63L and N71D and Y129M and P168S and Q184L and K244L and S258D and G259P; and P19E and T38E and K63L and N71D and Y129M and P168S and Q184L and K244L and S258D and N261R; wherein the amino acid positions of the variant or recombinant polypeptide or active fragment thereof are numbered by correspondence with the amino acid sequence of SEQ ID NO: 24.

In various embodiments, said two or more modifications are selected from P19E and T38E and K63L and N71D and Y129M and Q184L and K244L and S258D and N261R; P19E and T38E and N67D and N97D and Y129M and P168S and Q184L and K244L and S258D and N261R; P19E and K63L and N71D and N97D and Y129M and Q184L and G225C and K244L and S258D and G259P; P19E and T38E and N67D and N71D and N97D and Y129M and F167Y and Q184L and A217P and K244L and S258D and N261R; P19E and T38E and N67D and Y129M and P168S and Q184L and K244L and S258D and N261R; P19E and N67D and N97D and Y129M and P168S and Q184L and K244L; P19E and T38E and N67D and N97D and Q184L and A217P and G225C and T228V and Y235L and K244L and S258D and N261R; N10T and P19E and G28 S and S30T and T38E and N67D and N71D and N97D and Y129M and P168S and Q184L and G225C and Y235L and K244L and S258D and N261R and Z298.01Q; P19E and T38E and S59V and L60Q and K63R and N67D and N97D and V103I and Y 129M and K143Q and F167 Y and Q184L and G225C and Y235L and K244L and S258D and N261R and Z298.01Q; P19E and T38E and S59V and L60Q and K63L and K70R and N71D and Q78D and K80T and N97D and E111D and Y129M and Q184L and G225C and T228V and Y235L and K244L and S258D and N261R and Z298.01Q; N10T and P19E and T38E and N67D and Q78D and K80T and N97D and Y129M and K143Q and Q184L and A217P and G225C and T228V and Y235L and K244L and S258D and N261R and Z298.01Q; N10T and P19E and T38E and S59V and L60Q and K63L and N97D and V103I and Y129M and F167Y and Q184L and G225C and Y235L and K244L and S258D and N261R and Z298.01Q; P19E and S30T and T38E and S59V and L60Q and K63R and N67D and N97D and V103I and Y129M and F167Y and Q184L and G225C and T228V and Y235L and K244L and S258D and N261R and Z298.01Q; P19E and S30T and T38E and S59V and L60Q and K63R and N67D and Q78D and K80T and N97D and I124V and Y129M and K143Q and F167Y and Q184L and G225C and Y235L and K244L and S258D and N261R and Z298.01Q; N10S and P19E and S30T and T38E and S59V and L60Q and K63L and N67D and Q78H and K80T and 182M and N97D and Y129M and K143Q and F167Y and Q184L and G225C and Y235L and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; G4S and N10T and P19E and T38E and N67D and Q78D and K80T and N97D and Y129M and Q184L and G225C and T228V and Y235L and K244L and S258D and N261R and Z298.01Q; N10T and P19E and S30T and T38E and S59V and L60Q and K63L and K70R and N71D and Q78D and K80T and N97D and Y129M and T131A and F167Y and Q184L and G225C and Y235L and K244L and S258D and N261R and Z298.01Q; N10T and P19E and S30T and T38E and S59V and L60Q and K63L and K70R and N71D and Q78D and K80T and N97D and E111D and Y129M and P168S and Q184L and G225C and T228V and Y235L and K244L and S258D and N261R and Z298.01Q; P19E and S30T and T38E and S59V and L60Q and K63R and N67D and N97D and Y129M and P168S and Q184L and K214I and G225C and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; M1V and P19E and S30T and T38E and T62E and N67D and N71D and Q78D and N97D and Y129M and K143R and F167Y and P168S and Q184L and G225C and Y235L and K244L and S258D and N261R and T284A and Z298.01 Q; Y6E and N10T and P19E and G28S and S30T and T38E and K63L and N67D and N71D and N97D and E111S and Y129M and S135L and P168S and Q184L and G225C and T228V and Y235L and K244L and S258D and N261Q and D283S and Z298.01Q; N10T and P19E and S30T and T38E and S59V and L60Q and K63R and N67D and N71D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; M1L and N10 and P19E and G28A and S30T and T38E and K63L and N67D and N71D and Q78D and N97D and Y129M and A136L and P168A and Q184L and N213A and G225C and Y235L and K244L and S258D and N261R and Z298.01Q; P19E and T38E and S59V and K63R and N67D and N97D and V103I and Y129M and F167Y and Q184L and G225C and T228V and Y235L and K244L and S258D and N261R and Z298.01Q; N10 and P19E and G28A and S30T and T38E and K63R and N67D and N97D and Y129M and Q184L and G225C and T228V and Y235L and K244L and S258D and N261R and Z298.01Q; T3R and N10T and P19E and G28A and S30T and T38E and T62E and N67D and N71D and K93R and N97L and E111S and Y129M and D139M and P168S and Q184L and G225C and Y235L and K244L and S258D and N261Q and Z298.01Q; N10T and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; P19E and K63L and N71D and Y129M and P168S and Q184L and G225C and K244L; P19E and N67D and N71D and Q78D and K80T and N97D and Y129M and P168S and Q184L and K244L; P19E and T38E and N67D and Y129M and P168S and Q184L and T228V and K244L; P19E and T38E and N67D and Y129M and Q184L and K244L and S258D and N261R; P19E and K63L and N71D and Y129M and P168S and Q184L and K244L and S258D and N261R; P19E and T38E and K63L and N71D and Y129M and P168S and Q184L and K244L and S258D and G259P; and P19E and T38E and K63L and N71D and Y129M and P168S and Q184L and K244L and S258D and N261R; wherein the amino acid positions of the variant or recombinant polypeptide or active fragment thereof are numbered by correspondence with the amino acid sequence of SEQ ID NO: 24.

In various embodiments, said two or more modifications are selected from P19E and K63L and N67D and Q78D and K80T and N97D and Y129M and G225C and T228V and K244L; P19E and T38E and N67D and N71D and Q78D and K80T and N97D and Y129M and P168S and G225C and K244L and S258D and N261R; T38E and K63L and N71D and N97D and Y129M and Q184L and G225C and T228V and Q242L and K244L and S258D and N261R; P19E and K63L and N71D and N97D and Y129M and Q184L and G225C and K244L and S258D and G259P; N10 and T38E and S59V and L60Q and K63R and L66V and A68S and N74S and V75L and N97D and V103I and Y129M and F167Y and Q184L and A217P and G225C and Y235L and K244L and S258D and N261R and Z298.01Q; P19E and T38E and N67D and N97D and Q184L and A217P and G225C and T228V and Y235L and K244L and S258D and N261R; N10 and P19E and G28S and S30T and T38E and N67D and N71D and N97D and Y129M and P168S and Q184L and G225C and Y235L and K244L and S258D and N261R and Z298.01Q; P19E and T38E and S59V and L60Q and K63R and N67D and N97D and V103I and Y129M and K143Q and F167Y and Q184L and G225C and Y235L and K244L and S258D and N261R and Z298.01Q; P19E and T38E and N67D and N71D and Q78D and K80T and N97D and Y129M and P168S and G225C and T228V and K244L and S258D and N261R and Z298.01Q; P19E and T38E and S59V and L60Q and K63L and K70R and N71D and Q78D and K80T and N97D and E111D and Y129M and Q184L and G225C and T228V and Y235L and K244L and S258D and N261R and Z298.01Q; N10 and T38E and K63L and N71D and N97D and V103I and Y129M and F167Y and Q184L and G225C and T228V and Y235L and K244L and S258D and N261R and Z298.01Q; N10T and P19E and T38E and N67D and Q78D and K80T and N97D and Y129M and K143Q and Q184L and A217P and G225C and T228V and Y235L and K244L and S258D and N261R and Z298.01Q; N10T and P19E and T38E and S59V and L60Q and K63L and N97D and V103I and Y129M and F167Y and Q184L and G225C and Y235L and K244L and S258D and N261R and Z298.01Q; P19E and S30T and T38E and S59V and L60Q and K63R and N67D and N97D and V103I and Y129M and F167Y and Q184L and G225C and T228V and Y235L and K244L and S258D and N261R and Z298.01Q; P19E and S30T and T38E and S59V and L60Q and K63R and N67D and Q78D and K80T and N97D and I124V and Y129M and K143Q and F167Y and Q184L and G225C and Y235L and K244L and S258D and N261R and Z298.01Q; N10S and P19E and S30T and T38E and S59V and L60Q and K63L and N67D and Q78H and K80T and 182M and N97D and Y129M and K143Q and F167Y and Q184L and G225C and Y235L and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; G4S and N10T and P19E and T38E and N67D and Q78D and K80T and N97D and Y129M and Q184L and G225C and T228V and Y235L and K244L and S258D and N261R and Z298.01Q; N10T and P19E and S30T and T38E and S59V and L60Q and K63L and K70R and N71D and Q78D and K80T and N97D and Y129M and T131A and F167Y and Q184L and G225C and Y235L and K244L and S258D and N261R and Z298.01Q; N10T and P19E and S30T and T38E and S59V and L60Q and K63L and K70R and N71D and Q78D and K80T and N97D and E111D and Y129M and P168S and Q184L and G225C and T228V and Y235L and K244L and S258D and N261R and Z298.01Q; P19E and S30T and T38E and S59V and L60Q and K63R and N67D and N97D and Y129M and P168S and Q184L and K214I and G225C and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; M1V and P19E and S30T and T38E and T62E and N67D and N71D and Q78D and N97D and Y129M and K143R and F167Y and P168S and Q184L and G225C and Y235L and K244L and S258D and N261R and T284A and Z298.01Q; Y6E and N10T and P19E and G28S and S30T and T38E and K63L and N67D and N71D and N97D and E111 S and Y129M and S135L and P168S and Q184L and G225C and T228V and Y235L and K244L and S258D and N261Q and D283S and Z298.01Q; N10T and P19E and S30T and T38E and S59V and L60Q and K63R and N67D and N71D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; M1L and N10T and P19E and G28A and S30T and T38E and K63L and N67D and N71D and Q78D and N97D and Y129M and A136L and P168A and Q184L and N213A and G225C and Y235L and K244L and S258D and N261R and Z298.01Q; P19E and T38E and S59V and K63R and N67D and N97D and V103I and Y129M and F167Y and Q184L and G225C and T228V and Y235L and K244L and S258D and N261R and Z298.01Q; N10T and P19E and G28A and S30T and T38E and K63R and N67D and N97D and Y129M and Q184L and G225C and T228V and Y235L and K244L and S258D and N261R and Z298.01Q; and N10 and P19E and G28A and S30T and T38E and S59D and N67D and A68S and N71D and K93R and N97D and Y129M and K143Q and P168S and Q184D and G225C and Y235L and K244L and S258D and N261R and T284E and Z298.01Q; wherein the amino acid positions of the variant or recombinant polypeptide or active fragment thereof are numbered by correspondence with the amino acid sequence of SEQ ID NO:24.

In various embodiments, said two or more modifications are selected from P19E and T38E and K63L and N71D and Y129M and Q184L and K244L and S258D and N261R; T38E and K63L and N71D and N97D and Y129M and Q184L and G225C and T228V and Q242L and K244L and S258D and N261R; P19E and K63L and N71D and N97D and Y129M and Q184L and G225C and K244L and S258D and G259P; P19E and T38E and K63L and N71 D and Y129M and P168S and G225C and T228V and K244L and S258D and N261R; P19E and T38E and S59V and L60Q and K63L and K70R and N71 D and Q78D and K80T and N97D and E111D and Y129M and Q184L and G225C and T228V and Y235L and K244L and S258D and N261R and Z298.01Q; N10 and T38E and K63L and N71D and N97D and V103I and Y129M and F167Y and Q184L and G225C and T228V and Y235L and K244L and S258D and N261R and Z298.01Q; N10T and P19E and S30T and T38E and S59V and L60Q and K63L and K70R and N71D and Q78D and K80T and N97D and Y129M and T131A and F167Y and Q184L and G225C and Y235L and K244L and S258D and N261R and Z298.01Q; N10T and P19E and S30T and T38E and S59V and L60Q and K63L and K70R and N71 D and Q78D and K80T and N97D and E111D and Y129M and P168S and Q184L and G225C and T228V and Y235L and K244L and S258D and N261R and Z298.01Q; Y6E and N10 and P19E and G28S and S30T and T38E and K63L and N67D and N71D and N97D and E111 S and Y129M and S135L and P168S and Q184L and G225C and T228V and Y235L and K244L and S258D and N261Q and D283S and Z298.01Q; N10T and P19E and S30T and T38E and S59V and L60Q and K63R and N67D and N71 D and N97D and V103I and Y129M and K143Q and P168S and Q184L and G225P and T228V and Y235L and K244L and S258D and N261R and Z298.01Q; A2S and P19E and G28S and S30T and T38E and K63R and N67D and N71D and N74E and K93R and N97D and Y129M and N150T and P168S and Q184L and N213A and G225C and Y235L and K244L and S258D and N261Q and Z298.01Q; M1L and N10 and P19E and G28A and S30T and T38E and K63L and N67D and N71D and Q78D and N97D and Y129M and A136L and P168A and Q184L and N213A and G225C and Y235L and K244L and S258D and N261R and Z298.01Q; P19E and K63L and N71D and Y129M and P168S and Q184L and G225C and K244L; P19E and K63L and N71D and Y129M and P168S and Q184L and K244L and S258D and N261R; P19E and T38E and K63L and N71D and Y129M and P168S and Q184L and K244L and S258D and G259P; K63L and N71D and Y129M and K143R and P168S and Q184L and G225C and T228V and K244L and S258D and G259P; and P19E and T38E and K63L and N71D and Y129M and P168S and Q184L and K244L and S258D and N261R; wherein the amino acid positions of the variant or recombinant polypeptide or active fragment thereof are numbered by correspondence with the amino acid sequence of SEQ ID NO: 24.

In various embodiments, said two or more modifications are selected from P19E and T38E and N67D and N97D and Q184L and A217P and G225C and T228V and Y235L and K244L and S258D and N261R; P19E and T38E and S59V and L60Q and K63L and K70R and N71 D and Q78D and K80T and N97D and E111D and Y129M and Q184L and G225C and T228V and Y235L and K244L and S258D and N261R and Z298.01Q; N10T and T38E and K63L and N71D and N97D and V103I and Y129M and F167Y and Q184L and G225C and T228V and Y235L and K244L and S258D and N261R and Z298.01Q; N10T and P19E and T38E and N67D and Q78D and K80T and N97D and Y129M and K143Q and Q184L and A217P and G225C and T228V and Y235L and K244L and S258D and N261R and Z298.01Q; P19E and S30T and T38E and S59V and L60Q and K63R and N67D and N97D and V103I and Y129M and F167Y and Q184L and G225C and T228V and Y235L and K244L and S258D and N261R and Z298.01Q; N10T and P19E and S30T and T38E and S59V and L60Q and K63R and N67D and N97D and Y129M and K143Q and P168S and Q184L and G225C and T228V and Y235L and K244L and S258D and N261R and Z298.01Q; G4S and N10T and P19E and T38E and N67D and Q78D and K80T and N97D and Y129M and Q184L and G225C and T228V and Y235L and K244L and S258D and N261R and Z298.01Q; N10T and P19E and S30T and T38E and S59V and L60Q and K63L and K70R and N71D and Q78D and K80T and N97D and E111D and Y129M and P168S and Q184L and G225C and T228V and Y235L and K244L and S258D and N261R and Z298.01Q; N10T and P19E and S30T and T38E and S59V and L60Q and K63R and N67D and N97D and Y129M and K143Q and P168S and Q184L and G225P and T228V and Y235L and K244L and S258D and N261R and Z298.01Q; Y6E and N10T and P19E and G28S and S30T and T38E and K63L and N67D and N71D and N97D and E111S and Y129M and S135L and P168S and Q184L and G225C and T228V and Y235L and K244L and S258D and N261 Q and D283 S and Z298.01Q; N10T and P19E and S30T and T38E and S59V and L60Q and K63R and N67D and N71 D and N97D and V103I and Y129M and K143Q and P168S and Q184L and G225P and T228V and Y235L and K244L and S258D and N261R and Z298.01Q; P19E and T38E and S59V and K63R and N67D and N97D and V103I and Y129M and F167Y and Q184L and G225C and T228V and Y235L and K244L and S258D and N261R and Z298.01Q; N10T and P19E and G28A and S30T and T38E and K63R and N67D and N97D and Y129M and Q184L and G225C and T228V and Y235L and K244L and S258D and N261R and Z298.01Q; wherein the amino acid positions of the variant or recombinant polypeptide or active fragment thereof are numbered by correspondence with the amino acid sequence of SEQ ID NO: 24.

In various embodiments, said two or more modifications are P19E and S30T and T38E and S59V and L60Q and K63R and N67D and N97D and V103I and Y129M and F167Y and Q184L and G225C and T228V and Y235L and K244L and S258D and N261R and Z298.01Q; wherein the amino acid positions of the variant or recombinant polypeptide or active fragment thereof are numbered by correspondence with the amino acid sequence of SEQ ID NO: 24. Such mannanase is a preferred enzyme according to the invention, in particular if the rest of the amino acid sequence of SEQ ID NO:24 remains unchanged.

In various embodiments, said variant or recombinant polypeptide or active fragment thereof, as described above, further comprises one or more motifs selected from:

(i) WXₐKNDLXXAI (SEQ ID NO: 25) motif at positions 31-40, wherein Xₐ is F or Y and X is any amino acid;

(ii) LDXXXGPXGXLT (SEQ ID NO: 26) motif at positions 263-274, wherein X is any amino acid;

(iii) LDX₁V/AT/AGPX₂GX₃LT (SEQ ID NO: 27) motif at positions 263-274, wherein X₁ is an M or L, X₂ is N, A or S and X₃ is S, T or N; and

(iv) LDM/LATGPN/AGS/TLT (SEQ ID NO: 28) motif at positions 263-274, wherein the amino acid positions of the variant or recombinant polypeptide or active fragment thereof are numbered by correspondence with the amino acid sequence of SEQ ID NO: 24.

In various embodiments described herein, the variant, or recombinant polypeptide or active fragment thereof is not ACU308431, ETT37549, WP_036608478, WP_036670707, WP_017688745, WP_053782127, AAX87003, WP_046227931, WP_024633848, WP_017813111, PspMan9, AEX60762, WP_046214462, YP_003868989, YP_003944884, WP_017427981, AAX87002, WP_009593769, YP_006190599, or WP_019912481, or, optionally, PamMan2, PamMan3, PtuMan2, or PpaMan2.

In various embodiments, the mannanase variant or a recombinant polypeptide or an active fragment thereof comprises an amino acid sequence having at least 80% amino acid sequence identity to the amino acid sequence of SEQ ID NO: 29.

In various embodiments, the mannanase variant or recombinant polypeptide or active fragment thereof is derived from a reference polypeptide, wherein said reference polypeptide is selected from SEQ ID NOs: 24, 30, 31, 32, 33, 34, and 35(162), wherein the mannanase variant or recombinant polypeptide or active fragment thereof preferably has at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity with the amino acid sequence of said reference polypeptide.

In various embodiments, said reference polypeptide is a GH5 mannanase and said mannanase variant or recombinant polypeptide or active fragment thereof is optionally a GH5 mannanase or an endo-P-mannanase. The mannanase variant or recombinant polypeptide or active fragment thereof has mannanase activity, preferably in the presence of a surfactant and/or a protease, and/or may have one or more improved properties when compared to a reference polypeptide; wherein the improved property is selected from improved stability in the presence of protease, improved stability in detergent or buffer; and improved cleaning performance.

The improved property may be (i) improved stability in detergent, where said mannanase variant or recombinant polypeptide or active fragment thereof retains at least 10%, 20%, 30%, 40% or 50% residual mannanase activity at a temperature of about 40° C. to about 70° C., about 45° C. to about 65° C., about 50° C. to about 60° C., about 60° C. to about 70° C., or about 56° C. for a time period of at least 5 minutes; (ii) improved stability in the presence of protease, wherein said mannanase variant or recombinant polypeptide or active fragment thereof retains at least 50% mannanase activity in the presence of a protease and/or a surfactant for at least 15 days or from about 15 to about 40 days; and/or (iii) improved aged cleaning performance, wherein said mannanase variant or recombinant polypeptide or active fragment thereof has at least 15% remaining cleaning activity after 7 hours or at least 11% remaining cleaning activity after 9 hours.

In various embodiments, the mannanase variant or recombinant polypeptide or active fragment thereof does not further comprise a carbohydrate-binding module.

In various other embodiments, the mannanase is derived from Bacillus hemicellulosilyticus, Bacillus clausii or Virgibacillus soli or a variant thereof. Such mannanase variants comprise amino acid sequences which have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence according to SEQ ID NO:39, 40 or 41.

Suitable mannanases are commercially available, for example from Danisco/DuPont under the tradename Preferenz® M100. Further suitable mannanases are available under the tradename Biotouch® M7 from AB Enzymes or Mannaway® 4.0L or Mannaway® 200L from Novozymes.

Polypeptides Having Amylase Activity

An amylase is an enzyme that hydrolyses starch into sugars. Amylases that are used in accordance with the present invention are described in the following.

In various embodiments, the amylase is a (recombinant) variant of a parent alpha-amylase comprising: a mutation at an amino acid residue corresponding to R375, and optionally S360; and at least one mutation, and optionally at least two mutations, at an amino acid residue, or residues, corresponding to an amino acid residue selected from the group consisting of N126, F153, T180, E187, and I203; wherein the variant alpha-amylase or the parent alpha-amylase has at least 60%, at least 70%, at least 80%, at least 85%, 86%, 87%, 88%, 89%, 90%, 91%, 92%, 93%, 94%, 95%, 96%, 97%, 98%, or 99% amino acid sequence identity relative to SEQ ID NO: 36, which is used for numbering; and wherein the variant preferably has increased low pH stability and/or starch liquifaction activity, compared to the parent alpha-amylase or a reference alpha-amylase differing from the variant α-amylase only by the absence of the mutations.

In various embodiments, the variant alpha-amylase comprises the mutation R375Y, and optionally S360A; and at least one mutation, and optionally at least two mutations, at an amino acid residue, or residues, corresponding to an amino acid residue selected from the group consisting of N126Y, F153W, T180H, T180D, E187P, and I203Y, using the positional numbering of SEQ ID NO: 36.

In various embodiment, the above amylase variant comprises the mutations R375Y and S360A, using positional numbering according to SEQ ID NO: 36. The variant alpha-amylase may preferably further comprise the mutations N126Y, F153W, T180H, and E187P, using the positional numbering of SEQ ID NO: 36.

In various embodiments, the above variant alpha-amylase further comprises a mutation at a position selected from the group consisting of A275, T89, S92 and Y301, using the positional numbering of SEQ ID NO: 36.

In various embodiments, the above variant alpha-amylase further comprises a deletion of at least one amino acid residue corresponding to R178, G179, T180, and G181, using the positional numbering of SEQ ID NO: 36.

In various embodiments, the above variant alpha-amylase further comprises deletions of amino acid residues corresponding to R178 and G179, or T180 and G181, using the positional numbering of SEQ ID NO: 36.

In various embodiments, the above variant alpha-amylase further comprises a mutation at an amino acid residue corresponding to G476, G477, E132, Q167, A277, R458, T459, and/or D460, using the positional numbering of SEQ ID NO: 36.

In various embodiments, the above variant alpha-amylase is derived from a parental amylase from a Cytophaga species and/or not from a Bacillus species.

Also encompassed are (recombinant) variants of a parent alpha-amylase comprising: a mutation at at least one, and optionally a plurality, of amino acid residues corresponding to position T38, N126, F153, E187, 1203, G476, and G477; and, optionally at least one mutation at an amino acid residue corresponding to R178, G179, T180, and G181, wherein the variant alpha-amylase or the parent alpha-amylase has at least 60%, at least 70%, at least 80%, at least 85%, 86%, 87%, 88%, 89%, 90%, 91%, 92%, 93%, 94%, 95%, 96%, 97%, 98%, or 99% amino acid sequence identity relative to SEQ ID NO:36, which is used for numbering; and wherein the variant preferably has increased detergent stability and/or cleaning performance compared to the parent alpha-amylase or a reference alpha-amylase differing from the variant α-amylase only by the absence of the mutations. Said variant alpha-amylase may comprise at least one, and optionally a plurality, of the mutations T38N, N126Y, F153W, E187P, I203Y, G476K, and G477E, using SEQ ID NO:36 for numbering. It may further comprise a mutation at position T129, for example the mutation T1291. Said variant α-amylase may further comprise deletions of amino acid residues corresponding to R178 and G179, or T180 and G181, using SEQ ID NO: 1 for numbering, and/or a mutation at an amino acid residue corresponding to E132, Q167, A277, R458, T459, and/or D460, using SEQ ID NO:36 for numbering. The variant alpha-amylase may lack a mutation at an amino acid residue corresponding to position N88, N134, and/or L171, using SEQ ID NO:36 for numbering. The parental alpha-amylase may be from a Cytophaga species and/or not from a Bacillus species.

In various embodiments, the amylase is a (recombinant) variant of a parent alpha-amylase comprising:

iv) a mutation at an amino acid residue E187 using SEQ ID NO:36 for numbering; and at least one mutation at an amino acid residue selected from the group consisting of N126, Y150, F153, L171, T180, and I203 using SEQ ID NO:36 for numbering; wherein the variant alpha-amylase has at least 70% amino acid sequence identity to SEQ ID NO:36; or

v) a mutation at an amino acid residue E186 using SEQ ID NO:37 for numbering; and at least one mutation at an amino acid residue selected from the group consisting of N125, Y149, F152, L170, D179, and L202 using SEQ ID NO:37 for numbering; wherein the variant alpha-amylase has at least 70% amino acid sequence identity to SEQ ID NO: 37; or

vi) a mutation at an amino acid residue E189 using SEQ ID NO:38 for numbering; and at least one mutation at an amino acid residue selected from the group consisting of N128, Y152, F155, L173, T182, and L205 using SEQ ID NO:38 for numbering; wherein the variant alpha-amylase has at least 70% amino acid sequence identity to SEQ ID NO:38;

wherein the variant preferably has increased thermostability, detergent stability, starch liquefaction activity, and/or cleaning performance compared to the parent alpha-amylase or a reference alpha-amylase differing from the variant alpha-amylase only by the absence of the mutations.

In various embodiments, the variant alpha-amylase of i) above comprises:

(a) at least two mutations at amino acid residues N126, Y150, F153, L171, and I203, using SEQ ID NO: 36 for numbering; and/or

(b) a deletion of at least one amino acid residue selected from R178, G179, T180, and G181, using SEQ ID NO:36 for numbering; and/or

(c) deletions of amino acid residues R178 and G179, or T180 and G181 using SEQ ID NO:36 for numbering; and/or

(d) a mutation at amino acid residue G476 and/or G477, using SEQ ID NO:36 for numbering; and/or

(e) a mutation in an amino acid residue selected from the group consisting of E132, Q167, T180, and A277, using SEQ ID NO:36 for numbering; and/or

(f) a mutation in an amino acid residue selected from the group consisting of R458, T459, and D460, using SEQ ID NO:36 for numbering; and/or

(g) a mutation in amino acid residue T180, using SEQ ID NO:36 for numbering; and/or

(h) a mutation in an amino acid residue selected from the group consisting of 6, 7, 8, 11, 14, 15, 20, 21, 23, 26, 27, 28, 37, 38, 39, 40, 42, 45, 46, 48, 49, 50, 51, 52, 53, 54, 58, 61, 62, 68, 70, 71, 72, 73, 79, 80, 81, 82, 84, 85, 87, 88, 89, 92, 93, 94, 95, 96, 97, 98, 101, 108, 111, 112, 113, 114, 115, 116, 117, 118, 120, 122, 123, 124, 126, 127, 129, 130, 131, 132, 133, 134, 136, 137, 138, 140, 142, 143, 144, 147, 148, 149, 150, 151, 152, 153, 154, 155, 156, 158, 159, 165, 167, 168, 170, 171, 172, 175, 176, 177, 180, 181, 182, 187, 190, 191, 193, 199, 200, 201, 203, 206, 208, 210, 211, 212, 214, 215, 216, 219, 221, 223, 225, 226, 227, 235, 238, 239, 240, 241, 242, 243, 245, 246, 247, 248, 249, 250, 252, 253, 254, 256, 257, 258, 260, 261, 262, 266, 267, 268, 269, 270, 271, 273, 276, 277, 279, 280, 282, 284, 285, 286, 288, 296, 299, 300, 301, 302, 303, 304, 307, 308, 310, 311, 312, 313, 316, 317, 318, 320, 321, 325, 327, 335, 338, 342, 348, 349, 352, 356, 357, 360, 362, 363, 368, 369, 377, 381, 382, 383, 384, 385, 388, 390, 392, 394, 395, 396, 397, 398, 400, 401, 402, 403, 404, 405, 407, 408, 410, 414, 415, 416, 418, 419, 420, 421, 422, 423, 424, 426, 428, 429, 430, 431, 434, 435, 436, 439, 441, 442, 444, 445, 446, 447, 448, 449, 450, 451, 454, 455, 457, 460, 461, 462, 463, 464, 465, 466, 467, 469, 470, 471, 473, 474, 475, 476, 477, 479, 480, 481, 482, 483, and 484, using SEQ ID NO:36 for numbering; and/or

(i) a combination of mutations selected from the group consisting of: E187P+I203Y+G476K, E187P+I203Y+G476K+R458N+T459S+D460T, T180D+E187P+I203Y+G476K, N126Y+T180D+E187P+I203Y+G476K, N126Y+T180D+E187P+I203Y+Y303D+N475E+G477Q, N126Y+E187P+G476K, N126Y+F153W+E187P+G476K, N126Y+F153W+E187P+G4726+G477R, N126Y+E187P+I203Y, N126Y+T180H+E187P+I203Y, N126Y+F153W+T180H+E187P+I203Y, N126Y+Y150H+F153W+L171N+E187P+I203Y, and N126Y+Y150H+F153W+L171N+T180H+E187P+I203Y, wherein the variant preferably has increased thermostability, detergent stability, stability starch liquifaction activity, or cleaning performance compared to the parent and wherein SEQ ID NO:36 is used for numbering.

In various embodiments, the variant alpha-amylase of ii) above (comprising a mutation at an amino acid residue E186 using SEQ ID NO:37 for numbering; and at least one mutation at an amino acid residue selected from the group consisting of N125, Y149, F152, L170, D179, and L202 using SEQ ID NO:37 for numbering; wherein the variant alpha-amylase has at least 70% amino acid sequence identity to SEQ ID NO: 37 comprises:

(a) at least two mutations at amino acid residues N125, Y149, F152, L170, and L202, using SEQ ID NO: 37 for numbering; and/or

(b) a deletion of at least one amino acid residue selected from R177, G178, D179, and G180, using SEQ ID NO: 37 for numbering; and/or

(c) deletions of amino acid residues R177 and G178, or D179 and G180 using SEQ ID NO: 37 for numbering; and/or

(d) a mutation at amino acid residue G472 and/or G473, using SEQ ID NO: 37 for numbering; and/or

(e) a mutation in an amino acid residue selected from the group consisting of T131, Q166, D179, and T276, using SEQ ID NO: 37 for numbering; and/or

(f) a mutation in an amino acid residue selected from the group consisting of R454, S455, and G456, using SEQ ID NO: 37 for numbering; and/or

(g) a mutation in amino acid residue D179, using SEQ ID NO: 37 for numbering; and/or

(h) a mutation in amino acid residue N205, using SEQ ID NO: 37 for numbering; and/or

(i) a mutation in an amino acid residue selected from the group consisting of T333G, A335S, and Q337E, using SEQ ID NO: 37 for numbering; and/or

(j) a mutation in an amino acid residue selected from the group consisting of 5, 6, 7, 10, 13, 14, 19, 20, 22, 25, 26, 27, 36, 37, 38, 39, 41, 44, 45, 47, 48, 49, 50, 51, 52, 53, 57, 60, 61, 67, 69, 70, 71, 72, 78, 79, 80, 81, 83, 84, 86, 87, 88, 91, 92, 93, 94, 95, 96, 97, 100, 107, 110, 111, 112, 113, 114, 115, 116, 117, 119, 121, 122, 123, 125, 126, 128, 129, 130, 131, 132, 133, 135, 136, 137, 139, 141, 142, 143, 146, 147, 148, 149, 150, 151, 152, 153, 154, 155, 157, 158, 164, 166, 167, 169, 170, 171, 174, 175, 176, 179, 180, 181, 186, 189, 190, 192, 198, 199, 200, 202, 205, 207, 209, 210, 211, 213, 214, 215, 218, 220, 222, 224, 225, 226, 234, 237, 238, 239, 240, 241, 242, 244, 245, 246, 247, 248, 249, 251, 252, 253, 255, 256, 257, 259, 260, 261, 265, 266, 267, 268, 269, 270, 272, 275, 276, 278, 279, 281, 283, 284, 285, 287, 295, 298, 299, 300, 301, 302, 303, 306, 307, 309, 310, 311, 312, 315, 316, 317, 319, 320, 324, 326, 334, 337, 341, 347, 348, 351, 355, 356, 359, 361, 362, 367, 368, 373, 377, 378, 379, 380, 381, 384, 386, 388, 390, 391, 392, 393, 394, 396, 397, 398, 399, 400, 401, 403, 404, 406, 410, 411, 412, 414, 415, 416, 417, 418, 419, 420, 422, 424, 425, 426, 427, 430, 431, 432, 435, 437, 438, 440, 441, 442, 443, 444, 445, 446, 447, 450, 451, 453, 456, 457, 458, 459, 460, 461, 462, 463, 465, 466, 467, 469, 470, 471, 472, 473, 475, 476, 477, 478, 479, and 480, using SEQ ID NO: 37 for numbering; and/or

(k) a combination of mutations selected from the group consisting of:

N125Y+E186P+T333G+A335S+Q337E+G472K

N125Y+F152W+E186P+T333G+A335S+Q337E+G472K

N125Y+F152W+E186P+N205D+T333G+A335S+Q337E+G472K,

using SEQ ID NO: 37 for numbering.

In various embodiments, the variant alpha-amylase of iii) above, comprises:

(a) at least two mutations at amino acid residues N128, Y152, F155, L173, and L205, using SEQ ID NO: 38 for numbering; and/or

(b) a deletion of at least one amino acid residue selected from R180, S181, T182, and G183, using SEQ ID NO: 38 for numbering; and/or

(c) deletions of amino acid residues R180 and S181, or T182 and G183 using SEQ ID NO: 38 for numbering; and/or

(d) a mutation at amino acid residue G475 and/or G476, using SEQ ID NO: 38 for numbering; and/or (e) a mutation in an amino acid residue selected from the group consisting of T134, E169, T182, and T279, using SEQ ID NO: 38 for numbering; and/or

(f) a mutation in an amino acid residue selected from the group consisting of R457, S458, and D459, using SEQ ID NO: 38 for numbering; and/or

(g) a mutation in amino acid residue T182, using SEQ ID NO: 38 for numbering; and/or

(h) a mutation in an amino acid residue selected from the group consisting of 8, 9, 10, 13, 16, 17, 22, 23, 25, 28, 29, 30, 39, 40, 41, 42, 44, 47, 48, 50, 51, 52, 53, 54, 55, 56, 60, 63, 64, 70, 72, 73, 74, 75, 81, 82, 83, 84, 86, 87, 89, 90, 91, 94, 95, 96, 97, 98, 99, 100, 103, 110, 113, 114, 115, 116, 117, 118, 119, 120, 122, 124, 125, 126, 128, 129, 131, 132, 133, 134, 135, 136, 138, 139, 140, 142, 144, 145, 146, 149, 150, 151, 152, 153, 154, 155, 156, 157, 158, 160, 161, 167, 169, 170, 172, 173, 174, 177, 178, 179, 182, 183, 184, 189, 192, 193, 195, 201, 202, 203, 205, 208, 210, 212, 213, 214, 216, 217, 218, 221, 223, 225, 227, 228, 229, 237, 240, 241, 242, 243, 244, 245, 247, 248, 249, 250, 251, 252, 254, 255, 256, 258, 259, 260, 262, 263, 264, 268, 269, 270, 271, 272, 273, 275, 278, 279, 281, 282, 284, 286, 287, 288, 290, 298, 301, 302, 303, 304, 305, 306, 309, 310, 312, 313, 314, 315, 318, 319, 320, 322, 323, 327, 329, 337, 340, 344, 350, 351, 354, 358, 359, 362, 364, 365, 370, 371, 376, 380, 381, 382, 383, 384, 387, 389, 391, 393, 394, 395, 396, 397, 399, 400, 401, 402, 403, 404, 406, 407, 409, 413, 414, 415, 417, 418, 419, 420, 421, 422, 423, 425, 427, 428, 429, 430, 433, 434, 435, 438, 440, 441, 443, 444, 445, 446, 447, 448, 449, 450, 453, 454, 456, 459, 460, 461, 462, 463, 464, 465, 466, 468, 469, 470, 472, 473, 474, 475, 476, 478, 479, 480, 481, 482, and 483, using SEQ ID NO:38 for numbering; and/or

(i) a combination of mutations selected from the group consisting of:

N128Y+E189P+G475R

F155W+E189P+G475R

T134E+T182H+E189P+G475R

N128Y+T134E+T182H+E189P+G475R

N128Y+F155W+E189P+G475R

T134E+F155W+T182H+E189P+G475R

N128Y+T134E+F155W+T182H+E189P+G475R

N128Y+T134H+F155W+T182D+E189P+G475R

N128Y+T134E+F155W+T182G+E189P+G457R, using SEQ ID NO: 38 for numbering.

In various embodiments, the afore-mentioned variant amylase of i), ii) and iii) are derived from parental alpha-amylase that are not from a Bacillus species; optionally wherein the parental alpha-amylase is from a Cytophaga species or a Paenibacillus species.

In various embodiments, the variant amylase of i), ii) or iii) as defined above originate from a parental alpha-amylase that has at least 70%, at least 80, 81, 82, 83, 84, 85, 86, 87, 88, 89, 90, 91, 92, 93, 94, 95, 96, 97, 98, or 99% amino acid sequence identity to the amino acid sequence of SEQ ID NO: 36, SEQ ID NO: 37, or SEQ ID NO: 38, respectively. In various embodiments, the variant alpha-amylase itself has said degree of sequence identity.

In various embodiments, the variant amylase of i) is a recombinant variant of a parent alpha-amylase of SEQ ID NO:36, said variant having mutations consisting of a combination of mutations E187P+I203Y+G476K+R458N+T459S+D460T and deletion of amino acid residues R178 and G179.

Commercially available amylases are, for example, Preferenz® S210 (from Danisco/DuPont).

Compositions

The invention relates to cleaning compositions comprising a dispersin and a carbohydrase, optionally in combination with one or more additional cleaning composition components, as per the appended claims.

One embodiment of the invention relates to a cleaning composition, as defined herein, comprising a dispersin, at least one carbohydrase and a cleaning component, wherein the carbohydrase is an amylase or a mannanase, as defined herein. The carbohydrase is any of the amylases or mannanases mentioned under the heading “Polypeptides having amylase or mannanase activity” respectively.

As described the carbohydrases may act synergistically with the dispersin in reduction, and removal of target substrate e.g. the PNAG of biofilm. It is believed that when the dispersins and carbohydrases are acting together, the biofilm and PNAG components are more effectively dispersed or removed. It is thus advantageous to formulate dispersins with carbohydrases such as amylases and mannanases in cleaning compositions for cleaning.

One aspect of the invention relates to a method of formulating a cleaning composition comprising adding a dispersin, at least one carbohydrase and a cleaning component, wherein the carbohydrase is an amylase or a mannanase, as defined herein. The invention further relates to a kit intended for deep cleaning, wherein the kit comprises a solution of an enzyme mixture comprising a dispersin and a carbohydrase, wherein the carbohydrase is an amylase or a mannanase, as defined herein.

In one aspect of the invention the carbohydrase is an amylase, as defined above.

In one aspect of the invention the carbohydrase is a mannanase, as defined above.

The dispersins to be formulated together with the carbohydrases or to be used together with the carbohydrases should be compatible with cleaning components. Dispersins are at present not standard ingredients in cleaning compositions. However, dispersins suitable for use in cleaning compositions have been identified, e.g. in WO 2017/186936, WO 2017/186937 and WO 2017/186943. These applications also mentioned that dispersins may be formulated with other enzymes e.g. carbohydrases. However, none of these applications indicate that the dispersins may have synergy with e.g. mannanases or amylases. Enzymes, such as dispersins, should not only be compatible with the cleaning components, the dispersins should also be compatible with other enzymes, which may be present in a typical cleaning composition. Surprisingly, it was found that carbohydrases such as amylases and mannanases not only are compatible but may even act synergistically in respect of complex stain such as biofilm and polysaccharide reduction and removal e.g. in cleaning. In one embodiment the combination of dispersin and carbohydrase, i.e. amylase or mannanase, provide an additive or even a synergistic effect defined as an effect over and above the sum of the effects of the enzymes taken individually.

Particularly useful dispersins may be those of microbial origin. One embodiment of the invention relates to a cleaning composition comprising a dispersin, a carbohydrase and at least one cleaning component, wherein the carbohydrase is as defined herein and wherein the dispersin is microbial, preferably obtained from bacteria or fungi. In one embodiment, the cleaning composition comprise a dispersin from bacteria. One embodiment of the invention relates to a cleaning composition, as defined herein, comprising a dispersin, a carbohydrase and at least one cleaning component, wherein the dispersin is obtained from Terribacillus, Curtobacterium, Aggregatibacter, Haemophilus, Actinobacillus, Lactobacillus, Staphylococcus, Neisseria, Otariodibacter, Lactococcus, Frigoribacterium, Basfia, Weissella, Macrococcus or Leuconostoc, preferably Terribacillus or Lactobacillus.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 2.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 3.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 4.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 5.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 6.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 7.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 8.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 9.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 10.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 11.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 12.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 13.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 14.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 15.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 16.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 17.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 18.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 19.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 20.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 21.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 22.

One embodiment of the invention relates to a cleaning composition, as defined herein, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 23.

One embodiment relates to a cleaning composition, as defined herein, comprising a Terribacillus dispersin and a mannase and/or amylase, wherein the mannanase and/or amylase is any one of those described above under the section “Polypeptides having mannanase/amylase activity”.

One embodiment relates to a cleaning composition, as defined herein, comprising a Curtobacterium dispersin and a mannase and/or amylase, wherein the mannanase and/or amylase is any one of those described above under the section “Polypeptides having mannanase/amylase activity”.

One embodiment relates to a cleaning composition, as defined herein, comprising a Aggregatibacter dispersin and a mannase and/or amylase, wherein the mannanase and/or amylase is any one of those described above under the section “Polypeptides having mannanase/amylase activity”.

One embodiment relates to a cleaning composition, as defined herein, comprising a Haemophilus dispersin and a mannase and/or amylase, wherein the mannanase and/or amylase is any one of those described above under the section “Polypeptides having mannanase/amylase activity”.

One embodiment relates to a cleaning composition, as defined herein, comprising a Actinobacillus dispersin and a mannase and/or amylase, wherein the mannanase and/or amylase is any one of those described above under the section “Polypeptides having mannanase/amylase activity”.

One embodiment relates to a cleaning composition, as defined herein, comprising a Lactobacillus dispersin and a mannase and/or amylase, wherein the mannanase and/or amylase is any one of those described above under the section “Polypeptides having mannanase/amylase activity”.

One embodiment relates to a cleaning composition, as defined herein, comprising a Staphylococcus dispersin and a mannase and/or amylase, wherein the mannanase and/or amylase is any one of those described above under the section “Polypeptides having mannanase/amylase activity”.

One embodiment relates to a cleaning composition, as defined herein, comprising a dispersin and a mannase and/or amylase, wherein the mannanase and/or amylase is any one of those described above under the section “Polypeptides having mannanase/amylase activity”, and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1. One embodiment relates to a cleaning composition, as defined herein, comprising a dispersin and a mannanase and/or amylase, wherein the dispersin comprises or consists of a polypeptide comprising or having the amino acid sequence shown in SEQ ID NO:1 and wherein the mannanase/amylase is any one of those described above under the section “Polypeptides having mannanase/amylase activity”. In alternative embodiments the dispersin has at least 60%, at least 65%, at least 70%, at least 75%, at least 80%, at least 85%, at least 90%, at least 95%, at least 98% or 100% sequence identity to the amino acid sequence shown in any one of SEQ ID Nos: 2-23 or comprises or consists of a polypeptide comprising or having the amino acid sequence set forth in any one of SEQ ID Nos. 2-23.

One embodiment of the invention relates to a cleaning composition, as defined herein, comprising a mannase and/or amylase, wherein the mannanase and/or amylase is any one of those described above under the section “Polypeptides having mannanase/amylase activity”, and a polypeptide having dispersin activity, wherein the dispersin polypeptide is selected from the group consisting of:
a) a polypeptide having at least 60%, at least 65%, at least 70%, at least 75% at least 80%, at least 85%, at least 90%, at least 95%, at least 98% or 100% sequence identity to the polypeptide shown in SEQ ID NO: 1,
b) a polypeptide having at least 60%, at least 65%, at least 70%, at least 75% at least 80%, at least 85%, at least 90%, at least 95%, at least 98% or 100% sequence identity to the polypeptide shown in SEQ ID NO: 2,
c) a polypeptide having at least 60%, at least 65%, at least 70%, at least 75% at least 80%, at least 85%, at least 90%, at least 95%, at least 98% or 100% sequence identity to the polypeptide shown in SEQ ID NO: 3,
d) a polypeptide having at least 60%, at least 65%, at least 70%, at least 75% at least 80%, at least 85%, at least 90%, at least 95%, at least 98% or 100% sequence identity to the polypeptide shown in SEQ ID NO: 4,
e) a polypeptide having at least 60%, at least 65%, at least 70%, at least 75% at least 80%, at least 85%, at least 90%, at least 95%, at least 98% or 100% sequence identity to the polypeptide shown in SEQ ID NO: 5,
f) a polypeptide having at least 60%, at least 65%, at least 70%, at least 75% at least 80%, at least 85%, at least 90%, at least 95%, at least 98% or 100% sequence identity to the polypeptide shown in SEQ ID NO: 6,
g) a polypeptide having at least 60%, at least 65%, at least 70%, at least 75% at least 80%, at least 85%, at least 90%, at least 95%, at least 98% or 100% sequence identity to the polypeptide shown in SEQ ID NO: 7,
h) a polypeptide having at least 60%, at least 65%, at least 70%, at least 75% at least 80%, at least 85%, at least 90%, at least 95%, at least 98% or 100% sequence identity to the polypeptide shown in SEQ ID NO: 8,
i) a polypeptide having at least 60%, at least 65%, at least 70%, at least 75% at least 80%, at least 85%, at least 90%, at least 95%, at least 98% or 100% sequence identity to the polypeptide shown in SEQ ID NO: 9,
j) a polypeptide having at least 60%, at least 65%, at least 70%, at least 75% at least 80%, at least 85%, at least 90%, at least 95%, at least 98% or 100% sequence identity to the polypeptide shown in SEQ ID NO: 10,
k) a polypeptide having at least 60%, at least 65%, at least 70%, at least 75% at least 80%, at least 85%, at least 90%, at least 95%, at least 98% or 100% sequence identity to the polypeptide shown in SEQ ID NO: 11,
l) a polypeptide having at least 60%, at least 65%, at least 70%, at least 75% at least 80%, at least 85%, at least 90%, at least 95%, at least 98% or 100% sequence identity to the polypeptide shown in SEQ ID NO: 12,
m) a polypeptide having at least 60%, at least 65%, at least 70%, at least 75% at least 80%, at least 85%, at least 90%, at least 95%, at least 98% or 100% sequence identity to the polypeptide shown in SEQ ID NO: 13,
n) a polypeptide having at least 60%, at least 65%, at least 70%, at least 75% at least 80%, at least 85%, at least 90%, at least 95%, at least 98% or 100% sequence identity to the polypeptide shown in SEQ ID NO: 14,
o) a polypeptide having at least 60%, at least 65%, at least 70%, at least 75% at least 80%, at least 85%, at least 90%, at least 95%, at least 98% or 100% sequence identity to the polypeptide shown in SEQ ID NO: 15,
p) a polypeptide having at least 60%, at least 65%, at least 70%, at least 75% at least 80%, at least 85%, at least 90%, at least 95%, at least 98% or 100% sequence identity to the polypeptide shown in SEQ ID NO: 16,
q) a polypeptide having at least 60%, at least 65%, at least 70%, at least 75% at least 80%, at least 85%, at least 90%, at least 95%, at least 98% or 100% sequence identity to the polypeptide shown in SEQ ID NO: 17,
r) a polypeptide having at least 60%, at least 65%, at least 70%, at least 75% at least 80%, at least 85%, at least 90%, at least 95%, at least 98% or 100% sequence identity to the polypeptide shown in SEQ ID NO: 18,
s) a polypeptide having at least 60%, at least 65%, at least 70%, at least 75% at least 80%, at least 85%, at least 90%, at least 95%, at least 98% or 100% sequence identity to the polypeptide shown in SEQ ID NO: 19,
t) a polypeptide having at least 60%, at least 65%, at least 70%, at least 75% at least 80%, at least 85%, at least 90%, at least 95%, at least 98% or 100% sequence identity to the polypeptide shown in SEQ ID NO: 20,
u) a polypeptide having at least 60%, at least 65%, at least 70%, at least 75% at least 80%, at least 85%, at least 90%, at least 95%, at least 98% or 100% sequence identity to the polypeptide shown in SEQ ID NO: 21,
v) a polypeptide having at least 60%, at least 65%, at least 70%, at least 75% at least 80%, at least 85%, at least 90%, at least 95%, at least 98% or 100% sequence identity to the polypeptide shown in SEQ ID NO: 22, and
w) a polypeptide having at least 60%, at least 65%, at least 70%, at least 75% at least 80%, at least 85%, at least 90%, at least 95%, at least 98% or 100% sequence identity to the polypeptide shown in SEQ ID NO: 23,

wherein the amylase/mannanase is any one of those described above under the sections “Polypeptides having mannanase/amylase activity”, and wherein the composition preferably comprises at least one cleaning component.

One embodiment relates to a cleaning composition, as defined herein, comprising a dispersin and a mannanase and/or amylase, wherein the dispersin comprises or consists of a polypeptide selected from the group of polypeptides comprising the amino acid sequence shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23 and wherein the mannanase/amylase comprises or consists of any one of the amino acid sequences described above under the sections “Polypeptides having mannanase/amylase activity”.

One embodiment relates to a composition, as defined herein, comprising
a) at least 0.001 ppm of at least one polypeptide having dispersin activity, wherein the dispersin is selected from the group consisting of:
   i. a dispersin obtained from Terribacillus;
   ii. a dispersin obtained from Curtobacterium;
   iii. a dispersin obtained from Aggregatibacter;
   iv. a dispersin obtained from Haemophilus;
   v. a dispersin obtained from Actinobacillus;
   vi. a dispersin obtained from Lactobacillus;
   vii. a dispersin obtained from or Staphylococcus;
   viii. a polypeptide having hexosaminidase activity selected from: a polypeptide having at least 60%, at least 70%, at least 75%, at least 80%, at least 85%, at least 90%, at least 95%, at least 98%, at least 99% or 100% sequence identity to the polypeptide shown in SEQ ID NO: 1, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 2, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 3, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 4, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 5, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 6, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 7, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 8, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 9, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 10, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 11, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 12, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 13, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 92, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 15, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 16, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 17, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 18, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 19, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 20, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 21, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 22, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 23, and
b) at least 0.01 ppm of at least one mannanase and/or amylase, wherein the mannanase and/or amylase is selected from any one of those described above under the section “Polypeptides having mannanase/amylase activity”, and, optionally,
c) at least one additional component e.g. a cleaning component, preferably selected from surfactants, builders, bleach components, polymers, dispersing agents and additional enzymes.

The mannanase/amylase and dispersin may be included in the cleaning composition of the present invention at a level of from 0.01 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, from 250 ppm to 500 ppm. These concentrations preferably refer to active protein concentrations relative to the total amount of the cleaning composition. Preferably all “ppm” concentrations given herein are by weight.

The dispersins above may be combined with mannanase/amylase to form a blend to be added to the wash liquor solution. The concentration of the dispersin in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm. The concentration of the mannanase and/or amylase in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm.

The dispersins may be combined with any of the mannanase/amylase described above to form a blend to be added to a composition according to the invention.

One embodiment relates to a cleaning composition, as defined herein, comprising a dispersin and a mannanase and/or amylase, wherein (i) the amount of dispersin in the composition is from 0.01 to 1000 ppm and (ii) the amount of mannanase is from 0.01 to 1000 ppm and/or the amount of amylase is from 0.01 to 1000 ppm.

In addition to the mannanase/amylase and dispersin, the cleaning composition may further comprise at least one cleaning component. One embodiment relates to a cleaning composition, as defined herein, comprising a dispersin, a mannanase and/or amylase and at least one cleaning component, wherein the cleaning component is selected from surfactants, preferably anionic and/or nonionic, builders and bleach components.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

One embodiment relates to a cleaning composition, as defined herein, comprising a dispersin, a carbohydrase, wherein the amount of dispersin in the composition is from 0.01 to 1000 ppm and the amount of carbohydrase is from 0.01 to 1000 ppm.

The invention relates to cleaning compositions comprising an enzyme combination of the present invention in combination with one or more additional cleaning composition component(s), as defined herein.

Generally, the choice of additional components is within the skill of the artisan and includes conventional ingredients, including the exemplary non-limiting components set forth below.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a functionality, this is not to be construed as a limitation, as a component may comprise additional functionalities as will be appreciated by the skilled artisan.

Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 0.1% to about 15%, such as about 1% to about 40%, or about 3% to about 20%, or about 3% to about 10%. “About”, as used herein in relation to a numerical value means said value±10%, preferably ±5%. “About 5 wt %” thus means from 4.5 to 5.5 wt %, preferably from 4.75 to 5.25 wt %. The surfactant(s) is chosen based on the desired cleaning application, and may include any conventional surfactant(s) known in the art. When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof. When included therein the detergent will usually contain from about 0.01% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof. When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Preferred anionic surfactants are sulphate surfactants and in particular alkyl ether sulphates, especially C9-C15 alcohol ether sulfates, preferably ethoxylates or mixed ethoxylates/propoxylates, such as those with 1 to 30 EO, C12-C15 primary alcohol ethoxylate, such as those with 1 to 30 EO, C8-C16 ester sulphates and C10-C14 ester sulphates, such as mono dodecyl ester sulphates. Non-limiting examples of anionic surfactants include sulfates and sulfonates, in particular, linear alkylbenzenesulfonates (LAS), in particular C12-C13 alkyl benzene sulfonates, isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof. The anionic surfactants are preferably added to the detergent in the form of salts. Suitable cations in these salts are alkali metal ions, such as sodium, potassium and lithium and ammonium salts, for example (2-hydroxyethyl) ammonium, bis(2-hydroxyethyl) ammonium and tris(2-hydroxyethyl) ammonium salts. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof. Commercially available nonionic surfactants include Plurafac™, Lutensol™ and Pluronic™ range from BASF, Dehypon™ series from Cognis and Genapol™ series from Clariant.

In various embodiments, said surfactant preferably comprises at least one alkyl ether sulfate. Preferred alkyl ether sulfates are those of formula (I)

R₁—O-(AO)ₙ-SO₃⁻X⁺ (I).

In formula (I) R¹ represents a linear or branched, substituted or unsubstituted alkyl group, preferably a linear, unsubstituted alkyl group, more preferably a fatty alcohol moiety. Preferred R¹ moieties are selected from the group consisting of decyl, undecyl, dodecyl, tridecyl, tetradecyl, pentadecyl, hexadecyl, heptadecyl, octadecyl, nonadecyl, eicosyl moieties and mixtures thereof, wherein those groups with an even number of carbon atoms are preferred. Particularly preferred R¹ moieties are derived from C₁₀-C₁₈ fatty alcohols, such as those derived from coconut oil alcohols, tallow fatty alcohols, lauryl, myristyl, cetyl or stearyl alcohol or from C₁₀-C₂₀ oxoalcohols.

AO represents an ethyleneoxide (EO) or propyleneoxide (PO) group, preferably an ethyleneoxide group. The index n represents an integer from 1 to 50, preferably from 1 to 20 and more preferably from 1 to 10. Particularly preferably, n is 1, 2, 3, 4, 5, 6, 7 or 8. X represents a monovalent cation or the n-th part of an n-valent cation, preferred are alkali metal cations, specifically Na⁺ and K⁺, most preferably Na⁺. Further cations X⁺ may be selected from NH₄⁺, ½ Zn²⁺, ½ Mg²⁺, Ca²⁺, Mn²⁺, and combinations thereof.

In various preferred embodiments, the detergent compositions comprise an alkyl ether sulfate selected from fatty alcohol ether sulfates of formula (II)

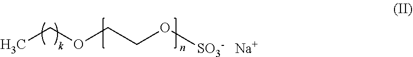

wherein k=9 to 19, and n=1, 2, 3, 4, 5, 6, 7 or 8. Preferred are C₁₀₋₁₆ fatty alcohol ether sulfates with 1-7 EO (k=9-15, n=1-7), such as the C₁₂₋₁₄ fatty alcohol ether sulfates with 1-3, particularly 2 EO (k=11-13, n=1-3 or 2), more particularly the sodium salts thereof. One specific embodiment thereof is lauryl ether sulfate sodium salt with 2 EO. The level of ethoxylation is an average value and can, for a specific compound, be an integer or fractional number.

In various embodiments, the surfactant comprises at least one alkyl benzene sulfonate. Said alkyl benzene sulfonate may be present alternatively to the above alkyl ether sulfate or, preferably, in addition to it.

Exemplary alkyl benzene sulfonates include, but are not limited to linear and branched alkyl benzene sulfonates, preferably linear alkyl benzene sulfonates. Exemplary compounds are those of formula (III)

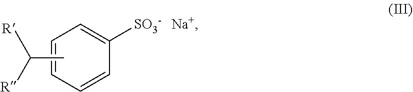

wherein R′ and R″ are independently H or alkyl and combined comprise 9 to 19, preferably 9 to 15 and more preferably 9 to 13 carbon atoms. Particularly preferred are dodecyl and tridecyl benzene sulfonates, in particular the sodium salts thereof.

In addition or alternatively, the compositions of the invention may further comprise one or more nonionic surfactants. Preferred nonionic surfactants are those of formula (IV)

R²—O-(AO)ₘ-H (IV),

wherein R² represents a linear or branched substituted or unsubstituted alkyl moiety, AO represents an ethylene oxide (EO) or propylene oxide (PO) group and m is an integer from 1 to 50.

In formula (IV) R² preferably represents a linear or branched, substituted or unsubstited alkyl group, preferably a linear, unsubstituted alkyl group, particularly preferred a fatty alcohol group. Preferred groups are R² are selected from decyl, undecyl, dodecyl, tridecyl, tetradecyl, pentadecyl, hexadecyl, heptadecyl, octadecyl, nonadecyl, eicosyl groups and combinations thereof, wherein those groups with an even number of carbon atoms are preferred. Particularly preferred are R² groups derived from C₁₂-C₁₈ fatty alcohols, such as coconut oil alcohol, tallow oil alcohol, lauryl, myristyl, cetyl or stearyl alcohol or from C₁₀-C₂₀ oxoalcohols.

AO represents an ethyleneoxide (EO) or propyleneoxide (PO) group, preferably an ethyleneoxide group. The index m represents an integer from 1 to 50, preferably from 1 to 20 and more preferably from 1 to 6. Particularly preferably, m is 1, 2, 3, 4 or 5, most preferably 3-5, as higher degrees of ethoxylation may negatively influence viscosity and stability.

In various preferred embodiments, the detergent compositions comprise an alkyl ether selected from fatty alcohol ethers of formula (V)

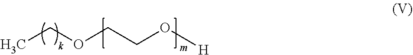

wherein k=11 to 19, m=1, 2, 3, 4, 5, 6, 7 or 8. Preferred are C₁₂₋₁₈ fatty alcohols with 1-6 EO (k=11-17, m=1-5 in formula (V)). More preferred are C₁₂₋₁₄ alcohols having 1-5 EO, most preferred are C₁₂₋₁₄ alkyl ethers with 3-5 EO, in particular lauryl ether with 5 EO.

The detergent compositions may further include other nonionic surfactants, such as alkyl glucosides of the general formula RO(G)ₓ, where R is a primary linear or 2-methyl-branched aliphatic radical containing 8 to 22 and preferably 12 to 18 carbon atoms and G stands for a glucose unit. The degree of oligomerization x, which indicates the distribution of monoglucosides and oligoglucosides, is a number of 1 to 10 and preferably a number of 1.2 to 1.4.

In various embodiments, the composition comprises at least two anionic surfactants, e.g. at least one alkyl ether sulfate and preferably at least one alkyl benzene sulfonate, and optionally an alkyl ether.

Suitable amphoteric surfactants comprise betains. Preferred betaines are the alkylbetaines, the alkylamidobetaines, the imidazolinium betaines, the sulfobetaines (INCI Sultaines) and the phosphobetaines. Examples of suitable betaines and sulfobetaines are the following compounds designated as INCI: almondamidopropyl betaines, apricotam idopropyl betaines, avocadamidopropyl betaines, babassuamidopropyl betaines, behenamide idopropyl betaines, behenyl betaines, betaines, canola idopropyl betaines, caprylic/capram idopropyl betaines, carnitines, cetyl betaines, Cocamidoethyl betaines, cocamidopropyl betaines, cocam idopropyl hydroxysultaines, cocobetaines, coco-hydroxysultaines, coco/oleam idopropyl betaines, coco-sultaines, decyl betaines, dihydroxyethyl oleyl glycinates, dihydroxyethyl soy glycinates, dihydroxyethyl stearyl glycinates, dihydroxyethyl tallow glycinates, dimethicones propyl PG Betaines, erucam idopropyl hydroxysultaines, hydrogenated tallow betaines, isostearam idopropyl betaines, lauram idopropyl betaines, lauryl betaines, lauryl hydroxysultaine, lauryl sultaines, milkamidopropyl betaines, minkam idopropyl betaines, myristamine idopropyl betaines, myristyl betaines, oleam idopropyl betaines, oleam idropy Hydroxysultain, Oleyl Betaine, Olivamidopropyl Betaine, Palmam Idopropyl Betaine, Palm Itam Idopropyl Betaine, Palmitoyl Carnitine, Palm Kernelamidopropyl Betaine, Polytetrafluoroethylene Acetoxypropyl Betaine, Ricinoleam Idopropyl Betaine, Sesamidopropyl Betaine, Soyamidopropyl Betaine, Stearam Idopropyl Betaine, Stearyl Betaine, Tallowam Idopropyl Betaine, Tallowamidopropyl Hydroxysultaine, Tallow Betaine, Tallow Dihydroxyethyl Betaine, Undecylenamidopropyl Betaine and Wheat Germamidopropyl Betaine. A preferred betaine is, for example, cocamidopropyl betaine (cocoamidopropylbetaine). The betaines are particularly preferred for dishwashing compositions, most preferably hand dishwashing detergent compositions.

Further suitable surfactants include the amine oxides. The amine oxides suitable in accordance with the invention include alkylamine oxides, in particular alkyldimethylamine oxides, alkylamidoamine oxides and alkoxyalkylamine oxides. Examples of suitable amine oxides are the following compounds designated as INCI: Almond amidopropylamine oxides, Babassu amidopropylamine oxides, Behenamine oxides, Cocamidopropyl Amine oxides, Cocamidopropylamine oxides, Cocamine oxides, Coco-Morpholine oxides, Decylamine oxides, Decyltetradecylamine oxides, Diaminopyrimidine oxides, Dihydroxyethyl C8-10 alkoxypropylamines oxides, Dihydroxyethyl C9-11 alkoxypropylamines oxides, dihydroxyethyl C12-15 alkoxypropylamines oxides, dihydroxyethyl cocamine oxides, dihydroxyethyl lauramine oxides, dihydroxyethyl stearamines oxides, dihydroxyethyl tallowamine oxides, hydrogenated palm kernel amine oxides, hydrogenated tallowamine oxides, hydroxyethyl hydroxypropyl C12-15 alkoxypropylamines oxides, isostearamidopropylamines Oxides, isostearamidopropyl morpholine oxides, lauram idopropylamine oxides, lauramine oxides, methyl morpholine oxides, milkamidopropyl amine oxides, mincamidopropylamine oxides, myristamine idopropylamine oxides, myristamine oxides, myristyl/cetyl amines Oxides, Oleam idopropylamine oxides, Oleamine oxides, OI ivam idopropylam ine oxides, Palmitamidopropylamine oxides, Palmitamine oxides, PEG-3 Lauramine oxides, Potassium dihydroxyethyl Cocamine oxides phosphates, Potassium Trisphosphonomethylamine oxides, Sesamidopropylamine oxides, Soyamidopropylamine oxides, Stearam idopropylam ine oxides, stearamines Oxides, Tallowam idopropylam ine oxides, Tallowamine oxides, Undecylenamidopropylamine oxides and Wheat Germam idopropylam ine oxides. A preferred amine oxide is, for example, cocamidopropylamine oxides (cocoamidopropylamine oxide).

For automatic dishwashing applications, low-foaming nonionic surfactants are preferably used, in particular alkoxylated, especially ethoxylated, low-foaming nonionic surfactants. With particular preference, the automatic dishwashing detergents contain nonionic surfactants from the group of the alkoxylated alcohols. Particular preference is given to nonionic surfactants which have a melting point above room temperature. Nonionic surfactants having a melting point above 20° C., preferably above 25° C., more preferably between 25 and 60° C. and especially between 26.6 and 43.3° C., are particularly preferred. Preferably used surfactants are those from the groups of alkoxylated nonionic surfactants, in particular the ethoxylated primary alcohols and mixtures of these surfactants with structurally more complex surfactants such as polyoxypropylene/polyoxyethylene/polyoxypropylene ((PO/EO/PO) surfactants). Such (PO/EO/PO) nonionic surfactants are also characterized by good foam control. Particularly preferred nonionic surfactants are those containing alternating ethylene oxide and different alkylene oxide units. Among these, in turn, surfactants with EO-AO-EO-AO blocks are preferred, with one to ten EO or AO groups before one block from the other group follows. Exemplary nonionic surfactants are those having a C9-alkyl group with 1 to 4 ethylene oxide units followed by 1 to 4 propylene oxide units, followed by 1 to 4 ethylene oxide units followed by 1 to 4 propylene oxide units. Preference is given in particular to end-capped, poly (oxyalkylated) nonionic surfactants with the end-cap being a linear or branched, saturated or unsaturated, aliphatic or aromatic hydrocarbon radical R having 1 to 30 carbon atoms. The alkyl groups may also comprise hydroxyl groups. The group of these nonionic surfactants include, for example, the C4-22 fatty alcohol (EO)₁₀₋₅₀-2-hydroxyalkyl ethers, in particular also the C8-12 fatty alcohol (EO)₂₂-2-hydroxydecyl ethers and the C4-22 fatty alcohol (EO)₄₀₋₈₀-2-hydroxyalkyl ethers.

Builders and Co-Builders

The detergent composition may contain about 0-65% by weight, such as about 5% to about 50%, such as from about 0.5 to about 20% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053 Generally and if not indicated otherwise, the builder may be preferably selected from citrate, carbonate, silicate, aluminosilicate (zeolite) and combinations thereof. Suitable builders also include phosphonates, polyphosphonates, bicarbonates, borates, and further polycarboxylates. Citrate builders, e.g., citric acid and soluble salts thereof (particularly sodium salt), are particularly suitable water-soluble organic builders. Citrates can be used in combination with zeolite, silicates like the BRITESIL types, and/or layered silicate builders. The builder and/or co-builder may be any chelating agent that forms water-soluble complexes with Ca and Mg. Any builder and/or co-builder known in the art for use in cleaning detergents may be utilized. Non-limiting examples of builders include zeolites, in particular zeolite A or P or X, carbonates such as sodium carbonate, soluble silicates such as sodium metasilicate, layered silicates (e.g., SKS-6 from Hoechst), and (carboxymethyl)inulin (CMI), and combinations thereof. Further non-limiting examples of builders include aminocarboxylates, aminopolycarboxylates, and alkyl- or alkenylsuccinic acid. Additional specific examples include 2,2′,2-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycine-N,N-diacetic acid (MGDA), glutamic acid-N,N-diacetic acid (GLDA), 1-hydroxyethane-1,1-diphosphonic acid, N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(sulfomethyl)aspartic acid (SMAS), N-(2-sulfoethyl)-aspartic acid (SEAS), N-(sulfomethylglutamic acid (SMGL), N-(2-sulfoethyl)-glutamic acid (SEGL), N-methyliminodiacetic acid (MIDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and N′-(2-hydroxyethyl)ethylenediamine-N,N,N′-triacetic acid (HEDTA), diethanolglycine (DEG), and combinations and salts thereof. Phosphonates suitable for use herein include 1-hydroxyethane-1,1-diphosphonic acid (HEDP), ethylenediaminetetrakis (methylenephosphonic acid) (EDTMPA), diethylenetriaminepentakis (methylenephosphonic acid) (DTMPA or DTPMPA or DTPMP), nitrilotris (methylenephosphonic acid) (ATMP or NTMP), 2-phosphonobutane-1,2,4-tricarboxylic acid (PBTC), hexamethylenediaminetetrakis (methylenephosphonic acid) (HDTMP). Particularly preferred are HEDP and DTPMP.

Suitable silicates are crystalline, layered sodium silicates of the general formula NaMSiₓO₂₊₁*yH₂O, wherein M is sodium or H, x a number of from 1.9 to 4 and y a number of from 0 to 20 and x is preferably 2, 3 or 4. Such silicates are for example disclosed in EP-A-0 164 514. Preferred are silicates in which M is sodium and is 2 or 3. Particularly preferred are β- and δ-sodium disilicate Na₂Si₂O₅*yH₂O.

Although not preferred, the compositions may also comprise phosphates, diphosphates (pyrophosphates) and/or triphosphates such as sodium triphosphate (STP or STPP). It is however preferred that all compositions disclosed herein are phosphate-free, i.e. do not contain deliberately added phosphate, in particular the phosphate content is below 1 wt %, more preferably less than 0.5 wt %, even more preferably less than 0.1 wt %, relative to the total weight of the composition. In alternative embodiments, the invention also relates to phosphate-free cleaning compositions in general that contain the polypeptides of the invention. In one aspect, the invention thus features a phosphate-free cleaning composition comprising any one or more of the polypeptides having hexosaminidase activity disclosed herein.

If not indicated otherwise, the composition may also contain 0-50% by weight, such as about 5% to about 30%, of a detergent co-builder. The composition may include a co-builder alone, or in combination with a builder, for example a zeolite builder. Non-limiting examples of co-builders include homopolymers of polyacrylates or copolymers thereof, such as poly (acrylic acid) (PAA) or copoly (acrylic acid/maleic acid) (PAA/PMA) or polyaspartic acid. Further exemplary builders and/or co-builders are described in, e.g., WO 09/102854, U.S. Pat. No. 5,977,053.

Preferred as co-builders are acrylate-containing water-soluble polymers, such as alkali metal salts of polyacrylic acid or polymethacrylic acid, for example those having a molecular weight MW in the range of 600 to 750,000 g/mol, as determined by gel permeation chromatography (GPC) according to DIN 55672-1:2007-08 with THE as an eluent.

Preferred polymers are polyacrylates with a molecular weight MW of 1,000 to 15,000 g/mol, more preferred, due to their solubility, are short-chain polyacrylates with a molecular weight MW of 1,000 to 10,000 g/mol, most preferred from 1,000 to 5,000 g/mol.

Preferred acrylates for use in the present invention are alkali metal salts of polymers of acrylic acid, preferably the sodium salts, in particular those with molecular weights in the range of 1,000 to 10,000 g/mol or 1,000 to 5,000 g/mol. Suitable acrylates are commercially available, for example under the tradename Acusol® from Dow Chemical. Suitable are also copolymers of acrylates, in particular those of acrylic acid and methacrylic acid, and acrylic acid or methacrylic acid and maleic acid.

In preferred embodiments, the compositions of the invention comprise a sulfopolymer, preferably a copolymer comprising an ethylenically unsaturated sulfonate/sulfonic acid as a co-monomer. Particularly suitable are monomers of allyl sulfonic acids, such as allyloxybenzene sulfonic acid and methallyl sulfonic acid. Particularly preferred sulfonic acid group-containing monomers are 1-acrylamido propane sulfonic acid-1,2-acrylamido-2-propanesulfonic acid, 2-acrylamido-2-methyl-1-propanesulfonic acid, 2-methacrylamido-2-methyl-1-propanesulfonic acid, 3-methacrylamido-2-hydroxy-propanesulfonic acid, allylsulfonic acid, methallylsulfonic acid, allyloxybenzenesulfonic acid, methallyloxybenzolsulfonsäure, 2-hydroxy-3-(2-propenyloxy) propanesulfonic acid, 2-methyl-2-propenl-sulfonic acid, styrenesulfonic acid, vinylsulfonic acid, 3-sulfopropyl, 3-sulfo-propyl, sulfomethacrylamide, sulfomethylmethacrylamide and mixtures of said acids or their water-soluble salts. The sulfopolymers are preferably copolymers of the afore-described monomers with unsaturated carboxylic acids, Especially preferred unsaturated carboxylic acids are acrylic acid, methacrylic acid, ethacrylic acid, chloroacrylic acid, alpha-cyanoacrylic acid, crotonic acid, alpha-phenyl-acrylic acid, maleic acid, maleic anhydride, fumaric acid, itaconic acid, citraconic acid, methylenemalonic acid, sorbic acid, cinnamic acid or mixtures thereof. Usable are of course also the unsaturated dicarboxylic acids.

Preferred are copolymers with acrylates, in particular with acrylic acid and methacrylic acid, and acrylic acid or methacrylic acid and maleic acid.

Such polymers are, for example, commercially available under the trade names Acusol® 590 or Acusol® 588 from Dow Chemical.

In one aspect of the invention, the cleaning compositions of the invention comprise a polypeptide as defined herein and at least one sulfopolymer, as defined above. Such compositions are preferably dishwashing compositions.

In one preferred embodiment, the builder is a non-phosphorus based builder such as citric acid and/or methylglycine-N,N-diacetic acid (MGDA) and/or glutamic-N,N-diacetic acid (GLDA) and/or salts thereof.

Bleaching Systems

The detergent may contain 0-30% by weight, such as about 1% to about 20%, such as from about 0.01 to about 10 wt % of a bleaching system. Any bleaching system comprising components known in the art for use in cleaning detergents may be utilized. Suitable bleaching system components include sources of hydrogen peroxide; sources of peracids; and bleach catalysts or boosters.

Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-KN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

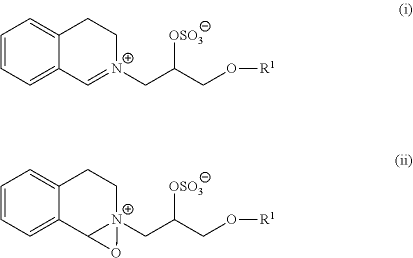

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Metal Care Agents

Metal care agents may prevent or reduce the tarnishing, corrosion or oxidation of metals, including aluminium, stainless steel and non-ferrous metals, such as silver and copper. Suitable examples include one or more of the following:

(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups (e.g., C1-C20-alkyl groups) and hydroxyl, thio, phenyl or halogen such as fluorine, chlorine, bromine and iodine.

(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate;

(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

Hydrotropes

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF.

Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

Enzymes

The detergent additive as well as the detergent composition may comprise one or more additional enzymes such as one or more proteases, lipases, cutinases, pectinases, arabinases, galactanases, xylanases, oxidases, e.g., a laccase, and/or peroxidases.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families. The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family. Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO09/021867, and Subtilisin lentus, Subtilisin Novo, subtilisin Carlsberg, Bacillus licheniformis, subtilisin BPN′, subtilisin 309, subtilisin 147 and subtilisin 168 and e.g. protease PD138 described in (WO93/18140).

Other useful proteases may be those described in WO01/016285 and WO02/016547. Examples of trypsin-like proteases are trypsin (e.g. of porcine or bovine origin) and the Fusarium protease described in WO94/25583 and WO05/040372, and the chymotrypsin proteases derived from Cellumonas described in WO05/052161 and WO05/052146. A further preferred protease is the alkaline protease from Bacillus lentus DSM 5483, as described for example in WO95/23221, and variants thereof which are described in WO92/21760, WO95/23221, EP1921147 and EP1921148. Examples of metalloproteases are the neutral metalloprotease as described in WO07/044993 (Proctor & Gamble/Genencor Int.) such as those derived from Bacillus amyloliquefaciens. Examples of useful proteases are the variants described in: WO89/06279 WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO: 1 of WO 2016/001449. More preferred the protease variants may comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease shown in SEQ ID NO: 1 of WO2016/001449, the Bacillus amylolichenifaciens protease (BPN′) shown in SEQ ID NO: 2 of WO2016/001449. The protease variants preferably have at least 80% sequence identity to SEQ ID NO: 1 or SEQ ID NO: 2 of WO 2016/001449. A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737. Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™ Purafect Prime®, Preferenz P110™, Effectenz P1000™ Purafect®™, Effectenz P1050™ Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), GeoBacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S). A peroxidase is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity. A suitable peroxidase includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. Preferably, the haloperoxidase is a vanadium haloperoxidase, i.e., a vanadate-containing haloperoxidase. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

A suitable oxidase includes in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular, a laccase derived from Coprinopsis cinerea, as disclosed in WO 97/08325; or from Myceliophthora thermophila, as disclosed in WO 95/33836.

Dispersants

The cleaning compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent

The cleaning compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives.

Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

Soil Release Polymers

The cleaning compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523. Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents

The cleaning compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose-based polymers described under soil release polymers above may also function as anti-redeposition agents.

Rheology Modifiers

The cleaning compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

Formulation of Detergent Products

The cleaning composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecularweight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

Granular Detergent Formulations

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The dispersin may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulate for the detergent industry is disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition as claimed and described herein in aqueous wash liquor, (ii) rinsing and/or drying the surface.

An embodiment of the invention relates to an enzyme granule/particle comprising the dispersin and at least one carbohydrase and a cleaning component, wherein the carbohydrase is an amylase or a mannanase, as defined herein. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm. The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606. The coating may be applied in an amount of at least 0.1% by weight of the core, e.g., at least 0.5%, 1% or 5%. The amount may be at most 100%, 70%, 50%, 40% or 30%. The coating is preferably at least 0.1 μm thick, particularly at least 0.5 μm, at least 1 μm or at least 5 μm. In a one embodiment, the thickness of the coating is below 100 μm. In another embodiment, the thickness of the coating is below 60 μm. In an even more particular embodiment the total thickness of the coating is below 40 μm. The coating should encapsulate the core unit by forming a substantially continuous layer. A substantially continuous layer is to be understood as a coating having few or no holes, so that the core unit it is encapsulating/enclosing has few or none uncoated areas. The layer or coating should be homogeneous in thickness. The coating can further contain other materials as known in the art, e.g., fillers, antisticking agents, pigments, dyes, plasticizers and/or binders, such as titanium dioxide, kaolin, calcium carbonate or talc. A salt coating may comprise at least 60% by weight w/w of a salt, e.g., at least 65%, at least 70%, at least 75%, at least 80%, at least 85%, at least 90%, at least 95% or at least 99% by weight w/w. The salt may be added from a salt solution where the salt is completely dissolved or from a salt suspension wherein the fine particles is less than 50 μm, such as less than 10 μm or less than 5 μm. The salt coating may comprise a single salt or a mixture of two or more salts. The salt may be water soluble and may have a solubility at least 0.1 grams in 100 g of water at 20° C., preferably at least 0.5 g per 100 g water, e.g., at least 1 g per 100 g water, e.g., at least 5 g per 100 g water. The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate.

Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{20° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{20° C.}=86%). Other examples include NaH₂PO₄, (NH₄)H₂PO₄, CuSO₄, Mg(NO₃)₂ and magnesium acetate. The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄.7H₂O), zinc sulfate heptahydrate (ZnSO₄.7H₂O), sodium phosphate dibasic heptahydrate (Na₂HPO₄-7H₂O), magnesium nitrate hexahydrate (Mg(NO₃)₂(6H₂O)), sodium citrate dihydrate and magnesium acetate tetrahydrate. Preferably the salt is applied as a solution of the salt, e.g., using a fluid bed.

One embodiment of the present invention provides a granule, which comprises:
(a) a core comprising a dispersin and a carbohydrase, wherein the carbohydrase is an amylase or a mannanase, as defined herein, and
(b) optionally a coating consisting of one or more layer(s) surrounding the core.

One embodiment of the invention relates to a granule, which comprises:
(a) a core comprising a dispersin and an amylase, as defined above, wherein the dispersin has at least 60%, at least 65%, at least 70%, at least 75%, at least 80%, at least 85%, at least 90%, at least 95%, at least 98% or 100% sequence identity to the amino acid sequence shown in any one of SEQ ID Nos: 1-23, and
(b) optionally a coating consisting of one or more layer(s) surrounding the core.

One embodiment of the invention relates to a granule, which comprises:
(a) a core comprising a dispersin and a mannanase, as defined above, wherein the dispersin has at least 60%, at least 65%, at least 70%, at least 75%, at least 80%, at least 85%, at least 90%, at least 95%, at least 98% or 100% sequence identity to the amino acid sequence shown in any one of SEQ ID Nos: 1-23, and
(b) optionally a coating consisting of one or more layer(s) surrounding the core.

Uses

The present invention is also directed to methods and use of the compositions of the invention in e.g. laundry/textile/fabric (House hold laundry washing, Industrial laundry washing) and hard surface cleaning (ADW, car wash, Industrial surface). The compositions of the invention comprise a blend of dispersin and carbohydrase, selected from an amylase and a mannanase, as defined herein, which effectively reduce or remove organic components, such as biofilm and components hereof e.g. mannan PNAG from surfaces such as textiles and hard surfaces e.g. dishes.

The compositions of the invention comprise a blend of dispersin and an amylase and/or a mannanase, and effectively reduce or remove organic components, such as mannan, starch, cellulose, xyloglucan, biofilm and components hereof e.g. PNAG from surfaces such as textiles and hard surfaces e.g. dishes. One embodiment of the invention relates to the use of a cleaning composition comprising a dispersin, a carbohydrase, selected from an amylase and a mannanase and at least one cleaning component for reduction or removal of stains such as biofilm and components hereof e.g. PNAG and at least one carbohydrase and a cleaning component, wherein the carbohydrase is an amylase or a mannanase, of an item, wherein the item is a textile or a hard surface.

One embodiment of the invention relates to the use of a cleaning composition comprising a dispersin, at least one carbohydrase and a cleaning component, wherein the carbohydrase is an amylase or a mannanase, as defined herein, for deep cleaning of an item, wherein the item is a textile or a surface.

One embodiment of the invention relates to the use of a composition comprising a dispersin and a carbohydrase, wherein the carbohydrase is an amylase or a mannanase, as defined herein, for reduction or removal of polysaccharide stains and/or compounds such as mannan, starch, cellulose, xyloglucan, biofilm and components hereof e.g. PNAG of an item. One embodiment of the invention relates to the use of a cleaning composition comprising a dispersin and a carbohydrase, wherein the carbohydrase is an amylase or a mannanase, as defined herein, for reduction or removal of polysaccharide stains and/or compounds such as mannan, starch, cellulose, xyloglucan and PNAG of an item such as textile. One embodiment of the invention relates to the use of a cleaning composition comprising a dispersin and a carbohydrase, wherein the carbohydrase is an amylase or a mannanase, as defined herein, for deep cleaning when the cleaning composition is applied in e.g. laundry process.

One embodiment of the invention relates to the use of a composition comprising a dispersin and carbohydrase, wherein the carbohydrase is an amylase or a mannanase, as defined herein, for reduction of redeposition or reduction of malodor. One embodiment of the invention relates to the use of a cleaning composition comprising a dispersin and carbohydrase, wherein the carbohydrase is an amylase or a mannanase, as defined herein, for reduction of redeposition or reduction of malodor.

One embodiment of the invention relates to the use of a composition comprising a dispersin and a mannanase for reduction of redeposition or reduction of malodor.

One embodiment of the invention relates to the use of a composition comprising a dispersin and an amylase for reduction of malodor.

One embodiment of the invention relates to the use of a cleaning composition comprising a dispersin and carbohydrase, wherein the carbohydrase is an amylase or a mannanase, as defined herein, for reduction of redeposition or reduction of malodor when the cleaning composition is applied in e.g. laundry process. One embodiment of the invention relates to the use of a cleaning composition comprising a dispersin and carbohydrase, wherein the carbohydrase is an amylase or a mannanase, as defined herein, for reduction of redeposition or reduction of malodor on an item e.g. textile. In one embodiment, the composition is an anti-redeposition composition.

One embodiment of the invention relates to the use of a cleaning composition comprising a dispersin and an amylase and/or mannanase, as defined herein, for deep cleaning of an item or reduction of redeposition or malodor. In various embodiments, the dispersin is selected from: a polypeptide having at least 60%, at least 70%, at least 75%, at least 80%, at least 85%, at least 90%, at least 95%, at least 98%, at least 99% or 100% sequence identity to the polypeptide shown in SEQ ID NO: 1, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 2, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 3, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 4, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 5, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 6, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 7, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 8, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 9, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 10, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 11, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 12, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 13, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 92, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 15, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 16, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 17, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 18, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 19, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 20, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 21, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 22, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 23.

The invention further relates to a method of deep cleaning of an item, wherein the item may be textile or hard surface preferably is a textile,

One embodiment of the invention relates to a method of deep cleaning on an item, comprising the steps of:
a) contacting the item with a cleaning composition according to the invention; and
b) and optionally rinsing the item, wherein the item is preferably a textile.

One embodiment of the invention relates to a method of deep cleaning on an item, comprising the steps of:
a) contacting the item with a solution comprising an enzyme mixture comprising a dispersin, a carbohydrase, wherein the carbohydrase is an amylase or a mannanase, as defined herein; and a cleaning component, wherein the cleaning component is selected from 0.1 to 50 wt % of at least one a surfactant; 0.5 to 30 wt % of at least one builder; and 0.01 to 20 wt % of at least one bleach component; and
b) and optionally rinsing the item, wherein the item is preferably a textile.

### Definitions

Biofilm is produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of proteins, and polysaccharides, such as PNAG. Biofilms may form on living or non-living surfaces. The microbial cells growing in a biofilm are physiologically distinct from planktonic cells of the same organism, which, by contrast, are single-cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community.

On laundry biofilm producing bacteria may e.g. be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. On hard surfaces biofilm producing bacteria may e.g. be found among the following species: Acinetobacter sp., Aeromicrobium sp., Brevundimonas sp., Microbacterium sp., Micrococcus luteus, Pseudomonas sp., Staphylococcus epidermidis, Staphylococcus aureus and Stenotrophomonas sp. In one aspect, the biofilm producing strain is Brevundimonas sp. In one aspect, the biofilm producing strain is Pseudomonas alcaliphila or Pseudomonas fluorescens. In one aspect, the biofilm producing strain is Staphylococcus aureus.

By the term “deep cleaning” is meant reduction, disruption or removal of components of organic matter, e.g. biofilm, such as polysaccharides, proteins, PNAG, soil or other components present in the organic matter.

Cleaning component: The cleaning component e.g. the detergent adjunct ingredient is different to the dispersin and carbohydrase. The precise nature of these additional cleaning components e.g. adjunct components, and levels of incorporation thereof, will depend on the physical form of the composition and the nature of the operation for which it is to be used. Suitable cleaning components e.g. adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Cleaning Composition: The term “cleaning composition” refers to compositions that find use in the removal of undesired compounds from items to be cleaned, such as textiles. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme blend of the invention, the detergent formulation may contain one or more additional enzymes (such as proteases, lipases, cutinases, endoglucanases, xyloglucanases, pectinases, pectin lyases, xanthanases, peroxidases, haloperoxygenases and catalases or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers, all of which are as defined herein.

The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides. Textile care benefits, which are not directly related to catalytic stain removal or prevention of redeposition of soils, are also important for enzyme detergency benefits. Examples of such textile care benefits are prevention or reduction of dye transfer from one textile to another textile or another part of the same textile (an effect that is also termed dye transfer inhibition or anti-backstaining), removal of protruding or broken fibers from a textile surface to decrease pilling tendencies or remove already existing pills or fuzz (an effect that also is termed anti-pilling), improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.”

The term “hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

The term “wash performance” is used as an enzyme's ability to remove stains present on the object to be cleaned during e.g. wash or hard surface cleaning.

The term “whiteness” is defined herein as a greying, yellowing of a textile. Loss of whiteness may be due to removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

By the term “malodor” is meant an odor which is not desired on clean items. The cleaned item should smell fresh and clean without malodors adhered to the item. One example of malodor is compounds with an unpleasant smell, which may be produced by microorganisms. Another example is unpleasant smells can be sweat or body odor adhered to an item which has been in contact with human or animal.

Another example of malodor can be the odor from spices, which sticks to items for example curry or other exotic spices which smells strongly.

The term “mature polypeptide” means a polypeptide in its final form following translation and any post-translational modifications, such as N-terminal processing, C-terminal truncation, glycosylation, phosphorylation, etc.

The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

The term “variant” means a polypeptide having the activity of the parent or precursor polypeptide and comprising an alteration, i.e., a substitution, insertion, and/or deletion, at one or more (e.g., several) positions compared to the precursor or parent polypeptide. A substitution means replacement of the amino acid occupying a position with a different amino acid; a deletion means removal of the amino acid occupying a position; and an insertion means adding an amino acid adjacent to and immediately following the amino acid occupying a position.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

Nomenclature: For purposes of the present invention, the nomenclature [E/Q] means that the amino acid at this position may be a glutamic acid (Glu, E) or a glutamine (Gln, Q). Likewise, the nomenclature [V/G/A/I] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

For an amino acid substitution, the following nomenclature is used: Original amino acid, position, substituted amino acid. For example, the substitution of a threonine at position 220 with alanine is designated as “T220A”. Multiple substitutions may be separated by addition marks (“+”), e.g., “T220A+G229V”, representing substitutions at positions 220 and 229 of threonine (T) with alanine (A) and glycine (G) with valine (V), respectively. Multiple substitutions may alternatively be listed with individual mutations separated by a space or a comma. Alternative substitutions in a particular position may be indicated with a slash (“/”). For example, substitution of threonine in position 220 with either alanine, valine or leucine many be designated “T220A/V/L”.

1. A cleaning composition comprising a dispersin, at least one carbohydrase and, optionally, a cleaning component, wherein the carbohydrase is an amylase and/or a mannanase selected from the group consisting of:
(I) a mannanase variant, or a recombinant polypeptide or an active fragment thereof comprising an amino acid sequence comprising two or more modifications selected from:
(1) (a) one or more substitutions at one or more positions selected from 1, 2, 3, 4, 6, 10, 19, 28, 30, 38, 59, 60, 61, 62, 63, 66, 67, 68, 70, 71, 74, 75, 78, 80, 82, 93, 97, 103, 111, 124, 129, 131, 135, 136, 139, 143, 150, 167, 168, 184, 213, 214, 217, 225, 228, 235, 242, 244, 258, 259, 261, 283, and 284; and
(b) an insertion at position 298; or
(2) one or more substitutions at one or more positions selected from 19, 38, 59, 67, 68, 71, 74, 97, 129, 167, 168, 184, 225, 228, 235, 242, 244, 258, and 261;
wherein the amino acid positions of the variant or recombinant polypeptide or active fragment thereof are numbered by correspondence with the amino acid sequence of SEQ ID NO: 24; and/or
(II) a mannanase variant comprising an amino acid sequence which has at least 90% sequence identity to the amino acid sequence according to SEQ ID NO:39, 40, or 41; and/or
(III) a variant of a parent alpha-amylase comprising:
a mutation at an amino acid residue corresponding to R375, and optionally 5360; and
at least one mutation at an amino acid residue, or residues, corresponding to an amino acid residue selected from the group consisting of N126, F153, T180, E187, and I203; wherein the variant alpha-amylase or the parent alpha-amylase has at least 90% amino acid sequence identity relative to SEQ ID NO: 36, which is used for numbering; and wherein the variant has increased low pH stability and/or starch liquifaction activity compared to the parent alpha-amylase or a reference alpha-amylase differing from the variant α-amylase only by the absence of the mutations; and/or
(IV) a variant of a parent alpha-amylase comprising:
i) a mutation at an amino acid residue E187 using SEQ ID NO: 36 for numbering; and at least one mutation at an amino acid residue selected from the group consisting of N126, Y150, F153, L171, T180, and 1203 using SEQ ID NO:36 for numbering;
wherein the variant alpha-amylase has at least 90% amino acid sequence identity to SEQ ID NO:36; or
ii) a mutation at an amino acid residue E186 using SEQ ID NO:37 for numbering; and at least one mutation at an amino acid residue selected from the group consisting of N125, Y149, F152, L170, D179, and L202 using SEQ ID NO:37 for numbering;
wherein the variant alpha-amylase has at least 90% amino acid sequence identity to SEQ ID NO: 37; or
iii) a mutation at an amino acid residue E189 using SEQ ID NO:38 for numbering;
and at least one mutation at an amino acid residue selected from the group consisting of N128, Y152, F155, L173, T182, and L205 using SEQ ID NO:38 for numbering;
wherein the variant alpha-amylase has at least 90% amino acid sequence identity to SEQ ID NO:38;
wherein the variant has increased thermostability, detergent stability, starch liquefaction activity, and/or cleaning performance compared to the parent alpha-amylase or a reference alpha-amylase differing from the variant alpha-amylase only by the absence of the mutations.

2. The cleaning composition according to claim 1, wherein said mannanase variant, or a recombinant polypeptide or active fragment thereof comprises two or more modifications selected from:
i) one or more substitutions at one or more positions selected from X1V, X1L, X2S, X3R, X4S, X6S, X6E, X10T, X10S, X19E, X28A, X28S, X30T, X38E, X59D, X59V, X60Q, X61W, X62E, X63R, X63L, X66V, X67D, X68S, X70R, X71D, X74E, X74S, X75L, X78D, X78H, X80T, X82M, X93R, X97D, X97L, X103I, X111D, X111S, X124V, X129M, X131A, X135L, X136L, X139M, X143Q, X143R, X150T, X167Y, X168A, X168S, X184D, X184L, X213A, X214I, X217P, X225C, X225P, X228V, X235L, X242L, X244L, X258D, X259P, X261Q, X261R, X283S, X284A, and X284E, and an insertion at position Z298.01Q; wherein X is any amino acid; or
ii) one or more substitutions at one or more positions selected from X19E, X38E, X59V, X67D, X68S, X71D, X74E, X74S, X97D, X97L, X129M, X167Y, X168A, X168S, X184D, X184L, X225C, X225P, X228V, X235L, X242L, X244L, X258D, X261Q, and X261R, wherein X is any amino acid;
wherein the amino acid positions of the variant or recombinant polypeptide or active fragment thereof are numbered by correspondence with the amino acid sequence of SEQ ID NO: 24.

3. The cleaning composition according to claim 2, wherein said two or more modifications are selected from P19E, S30T, T38E, S59V, L60Q, K63R, N67D, N97D, V103I, Y129M, F167Y, Q184L, G225C, T228V, Y235L, K244L, S258D, N261R, and Z298.01Q; wherein the amino acid positions of the variant or recombinant polypeptide or active fragment thereof are numbered by correspondence with the amino acid sequence of SEQ ID NO: 24.

4. The cleaning composition of claim 1, wherein the amylase is an alpha-amylase variant comprising: a mutation at at least one amino acid residue corresponding to position T38, N126, F153, E187, I203, G476, and G477; and, optionally at least one mutation at an amino acid residue corresponding to R178, G179, T180, and G181, wherein the variant alpha-amylase or the parent alpha-amylase has at least 90% amino acid sequence identity relative to SEQ ID NO:36, which is used for numbering; and further comprises a mutation at position T129, and further comprises deletions of amino acid residues corresponding to R178 and G179, or T180 and G181, using SEQ ID NO: 36 for numbering.

5. The cleaning composition according to claim 1, wherein the dispersin is microbial, preferably obtained from bacteria or fungi.

6. The cleaning composition according to claim 1, wherein the dispersin is obtained from Terribacillus, Curtobacterium, Aggregatibacter, Haemophilus, Actinobacillus, Lactobacillus, Staphylococcus, Neisseria, Otariodibacter, Lactococcus, Frigoribacterium, Basfia, Weissella, Macrococcus, or Leuconostoc.

7. The cleaning composition according to claim 1, wherein the dispersin catalyzes the hydrolysis of β-1,6-glycosidic linkages of N-acetyl-glucosamine polymers.

8. The cleaning composition according to claim 5, wherein the dispersin comprises a polypeptide selected from the group consisting of:
i) a polypeptide having at least 90% sequence identity to the amino acid sequence shown in SEQ ID NO: 1;
ii) a polypeptide having at least 90%, sequence identity to the amino acid sequence shown in SEQ ID NO: 2;
iii) a polypeptide having at least 90% sequence identity to the amino acid sequence shown in SEQ ID NO: 3;
iv) a polypeptide having at least 60%, at least 65%, at least 70%, at least 75%, at least 80%, at least 85%, at least 90%, at least 95%, at least 98% or 100% sequence identity to the amino acid sequence shown in SEQ ID NO: 4;
v) a polypeptide having at least 90% sequence identity to the amino acid sequence shown in SEQ ID NO: 5;
vi) a polypeptide having at least 90% sequence identity to the amino acid sequence shown in SEQ ID NO: 6;
vii) a polypeptide having at least 90% sequence identity to the amino acid sequence shown in SEQ ID NO: 7;
viii) a polypeptide having at least 90% sequence identity to the amino acid sequence shown in SEQ ID NO: 8;
ix) a polypeptide having at least 90% sequence identity to the amino acid sequence shown in SEQ ID NO: 9;
x) a polypeptide having at least 90% sequence identity to the amino acid sequence shown in SEQ ID NO: 10;
xi) a polypeptide having at least 90% sequence identity to the amino acid sequence shown in SEQ ID NO: 11;
xii) a polypeptide having at least 90% sequence identity to the amino acid sequence shown in SEQ ID NO: 12;
xiii) a polypeptide having at least 90% sequence identity to the amino acid sequence shown in SEQ ID NO: 13;
xiv) a polypeptide having at least 90% sequence identity to the amino acid sequence shown in SEQ ID NO: 14;
xv) a polypeptide having at least 90% sequence identity to the amino acid sequence shown in SEQ ID NO: 15;
xvi) a polypeptide having at least 90% sequence identity to the amino acid sequence shown in SEQ ID NO: 16;
xvii) a polypeptide having at least 90% sequence identity to the amino acid sequence shown in SEQ ID NO: 17;
xviii) a polypeptide having at least 90% sequence identity to the amino acid sequence shown in SEQ ID NO: 18;
xix) a polypeptide having at least 90% sequence identity to the amino acid sequence shown in SEQ ID NO: 19;
xx) a polypeptide having at least 90% sequence identity to the amino acid sequence shown in SEQ ID NO: 20;
xxi) a polypeptide having at least 90% sequence identity to the amino acid sequence shown in SEQ ID NO: 21;
xxii) a polypeptide having at least 90% sequence identity to the amino acid sequence shown in SEQ ID NO: 22; and
xxiii) a polypeptide having at least 90% sequence identity to the amino acid sequence shown in SEQ ID NO: 23.

9. The cleaning composition according to claim 1, wherein the dispersin comprises a polypeptide having at least 90% sequence identity to the amino acid sequence shown in SEQ ID NO: 17.

10. The cleaning composition according to claim 1, wherein an amount of dispersin in the composition ranges from 0.01 to 1000 ppm, and wherein the amount of amylase and/or mannanase ranges from 0.01 to 1000 ppm.

11. The cleaning composition according to claim 1, wherein the cleaning component is selected from surfactants, preferably anionic and/or nonionic, builders, and bleach components.

12. The cleaning composition of claim 1, wherein the composition
(a) is a solid, and further comprises
(a1) at least one zeolite builder;
(a2) at least one phosphonate builder;
(a3) at least one further enzyme; and
(a4) at least one polymer; or
(b) is a solid laundry detergent composition and further comprises
(b1) at least one silicate builder;
(b2) optionally carboxymethylcellulose;
(b3) at least one further enzyme;
(b4) optionally at least one soil release polymer; and
(b5) at least one bleaching system, comprising a bleaching agent, a bleach activator and a bleach catalyst; or
(c) is a liquid laundry detergent composition and further comprises
(c1) at least one surfactant;
(c2) optionally at least one phosphonate builder;
(c3) optionally at least at least one further enzyme; and
(c4) optionally at least one organic solvent; or
(d) is a liquid laundry detergent in unit dose form, and further comprises
(d1) water in an amount of up to 20 wt.-%;
(d2) optionally at least one bittering agent; and
(d4) optionally at least one polymer; or
(e) is a fabric finisher and further comprises
(e1) at least one softening silicone;
(e2) at least one perfume;
(e3) optionally polyquaternium 10 in an amount ranging from 0.1 to 20 wt.-%;
(e4) optionally polyquaternium 37 in an amount ranging from 0.1 to 20 wt.-%;
(e5) optionally a plant-based esterquat; and
(e6) optionally adipic acid, in an amount ranging from 0.1 to 20 wt.-%; or
(f) is an acidic cleaning agent and further comprises
(f1) plant-based or bio-based surfactants;
(f2) at least one acidic biocide; and
(f3) at least one soil release, water repellant or water spreading polymer; or
(g) is a neutral cleaning agent and further comprises
(g1) plant-based or bio-based surfactants;
(g2) at least one biocide; and
(g3) at least one soil release, water repellant or water spreading polymer; or
(h) is an alkaline cleaning agent and further comprises
(h1) plant-based or bio-based surfactants; or
(i) is a hand dishwashing agent and further comprises
(i1) at least one anionic surfactant;
(i2) at least one amphoteric surfactant;
(i3) at least one nonionic surfactant;
(i4) at least one further enzyme; or
(j) is an automatic dishwashing composition and further comprises
(j1) at least one builder selected from citrate, aminocarboxylates, and combinations thereof;
(j2) at least one phosphonate builder;
(j3) at least one nonionic surfactant;
(j4) at least one bleaching system comprising a bleaching agent, a bleach activator, and a bleach catalyst; and
(j5) at least one polymer selected from sulfopolymers, cationic polymers, polyacrylates, or combinations thereof; or
(k) further comprises
(k1) at least one sulfopolymer; or
(l) further comprises probiotics; or
(m) is in unit dose form and comprises at least 2 separate compartments; or
(n) is a phosphate-free composition.

13. (canceled)

14. A method of deep cleaning an item, wherein the method comprises:
contacting the item with a cleaning composition according claim 1; and optionally
rinsing the item, wherein the item is a textile.

15. The cleaning composition of claim 4, wherein the variant comprises one or more of the mutations T38N, N126Y, F153W, E187P, 1203Y, G476K, and G477E, using SEQ ID NO: 36 for numbering.

16. The cleaning composition of claim 5, wherein the dispersin is obtained from bacteria or fungi.

17. The cleaning composition of claim 11, wherein the surfactants are selected from anionic surfactants, nonionic surfactants, or both.


SEQUENCE LISTING (41 sequences)

SEQ ID NO: 1 - protein, 359 aa, Aggregatibacter actinomycetemcomitans
CVKGNSIHPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGIKYLQGLKSRQVDDEIDITNADSIAFMQSLMSEVIDIFGDTSQHFHIGGDEFGYSVESNHEFITYANKLSYFLEKKGLKTRMWNDGLIKSTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKANGDE

SEQ ID NO: 2 - protein, 346 aa, Haemophilus sputorum
QNSTKQSGLMLDISRRFYSVETIKQFIDDIAQANGTFLHLHFADHENYALESTFLNQRAENAIVQNGIYINPKTNKPFLTYEQIDQIIRYAQEKQIELIPEVDSPAHIKGILTLLRLEKGEDYVNQIALNQDELNLDSPESLTMMKTLVDEVCYIFGYSAQHFHIGGDEFNYASNFIRYVNALNQHINQKGLITRMWNDGLLQQNIDELDKNIEITYWSFDGDAQEKNDIVERRATRISLPTLLDKGFKALNYNSYYLYFIPKDNGNIATDAKFALNDLKQNWQLLRWDGNYETQPIQQAENLIGAAFSIWGEHAGKLSDDVIHQATSPLIQATIIQTNAKTTGPN

SEQ ID NO: 3 - protein, 352 aa, Actinobacillus suis
MNHSQIKEAGLTLDIARRFYPVETIKQFIDTIHHAGGTFLHLHFSDHENYALESTYLDQSEANAIVKDGTYYNPKTNKPFLTYKQIHDIIYYAKSKNIELVPEVDTPNHMTAIFRLLEAKHGKDYVKKLKSKMNDEEIDITNPESIEVIKTLIAEVIYIFGHASEHFHIGGDEFGYSVETNHEFISYVNTLNQFINEKGKITRIWNDGLIKNNLNQLNKNVEITYWSYDGDAQESQDIAERRKIRANLPELLENGFKVLNYNSYYLYFVPKGNANITHDSKYATEDVLNNWKLGLWDGQNKENMVENTKNIIGSSLSIWGERSGSLSSEVIEESTQDLLKAVIQKTNDPKSH

SEQ ID NO: 4 - protein, 352 aa, Actinobacillus capsulatus
MNHSQIKEAGLTLDIARRFYPVETIKQFIDTIHHAGGTFLHLHFSDHENYALESTYLDQLEANAIVKDGTYYNPTTNKPFLTYKQINDIIYYAKSKNIELVPEVDTPNHMTAIFRLLEAKHSKDYVKRLKSKMNDEEIDITNLESIEVIKTLIAEVIYIFGHASEHFHIGGDEFGYSVETNHEFITYVNTLNQFINNKGKITRIWNDGLIKNNLNQLNKNVEITYWSYDGDAQESQDIAERRKIRVNLPELLENGFKVLNYNSYYLYFVPKGNANITHDSKHATEDVLKNWKLGLWDGQNKENIVENTKNIIGSSLSIWGEHSGSLSSAVIEESTQELLKAVIQKTNDPKSH

SEQ ID NO: 5 - protein, 352 aa, Actinobacillus equuli
MNHSQIKEAGLTLDIARRFYPVETIKQFIDTIHHAGGTFLHLHFSDHENYALESSYLDQSEENAIVKDGTYYNPKTNKPFLTYKQIDDIIYYAKSKNIELVPEVDTPNHMTAIFNLLEIKHGEAYVKNLKSKMNDEEIDITNPESIEVIKTLIAEVIYIFGHASEHFHIGGDEFGYSVETNHEFISYVNTLNQFINEKGKITRIWNDGLIKNNLNQLNKNVEITYWSYDGDAQKSQDIAERRKIRADLPELLENGFKVLNYNSYYLYFVPKGNANITHDSKYATEDVLNNWKLGLWDGKNKENEVKNTKNIIGSSLSIWGERSGSLSSEVIEESTQDLLKAVIQKTNDPKSH

SEQ ID NO: 6 - protein, 359 aa, Aggregatibacter actinomycetemcomitans
CVKGNSIYPQKISTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSIAFMQSLMNEVIDIFGDTSQHFHIGGDEFGYSVESNHEFITYANKLSYFLEKKGLKTRMWNDGLIKSTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 7 - protein, 359 aa, Aggregatibacter actinomycetemcomitans
CVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 8 - protein, 351 aa, Actinobacillus pleuropneumoniae
MDLPKKESGLTLDIARRFYTVDTIKQFIDTIHQAGGTFLHLHFSDHENYALESSYLEQREENATEKNGTYFNPKTNKPFLTYKQLNEIIYYAKERNIEIVPEVDSPNHMTAIFDLLTLKHGKEYVKGLKSPYIAEEIDINNPEAVEVIKTLIGEVIYIFGHSSRHFHIGGDEFSYAVENNHEFIRYVNTLNDFINSKGLITRVWNDGLIKNNLSELNKNIEITYWSYDGDAQAKEDIQYRREIRADLPELLANGFKVLNYNSYYLYFVPKSGSNIHNDGKYAAEDVLNNWTLGKWDGKNSSNHVQNTQNIIGSSLSIWGERSSALNEQTIQQASKNLLKAVIQKTNDPKSH

SEQ ID NO: 9 - protein, 461 aa, Curtobacterium oceanosedimentum
ADRNTSAAEAAVTSIAPRATITGVAAISAATSSRTTVRTTLTLENRSGERESAADAWLYLAGGGARYALGHAPVRALAAGARATVRTTLRVPSRAPAGKYAVLACAGPYSKQACRTSGTTVTVGTAARARPETGVMLDVARAYYPVSLIEQYVDLLAEHGGGFLHLHLTDDQNVGIESAVLGQTPANAVLRNGVYTSRVTGRPFLSAAQARAISAYAAKRGIAIVPEVDSPGHMAAAFALLEARHGATWVDRIRSGESELDTSVPESATLAAELLREVTQTFPSSRTVHIGGDEWGADVSADERVGWMNAMAAAIGDREVWAWNDGIDRASVGRLDPRIHVTYWSFDGDTEDAAERRERRARRASATDLQRAGIDLLNYNSYYLYEVPTDLDPADSEYTVADLREHWSLRAWDGDSGARLAAPMSGAAVAIWGEDLDGAPSEALLRWSAPHVTAMIETAAS

SEQ ID NO: 10 - protein, 445 aa, Curtobacterium flaccumfaciens
DTAVSAVTVTKVTASTTGTVVRTTLKVENTAPVRKPASSVWLYLSAGTEKYTLGRVAVKALAAGSSTSVTAVRGTPSRAAAGKYSVLACAGAYSAKQCRTSTATVTTKPTKRARPETGVMLDVARAYYPVALIKRYIDLLADDGGRFLHLHLTDDQNVGIESTVLGQTPANADLDHGVYTSRVTHRPFLSAAQARTISAYGAERGVAIVPEIDTPGHMAAAFALLEAQHGTKWVDRIRSGENELDTSAPESLALAKKLYAEVQRTFPSSRTVHIGGDEWGDDVTAAQRVTWMNAMAAALDDREVWAWNDGIDRVAVGRLDPRIHVTYWSFDGDTEDAAERRERRARRASAVDLQQAGIDQLNYNSYYLYEVPTDLDPADSDYTVADLRENWSLRAWDGDSGSLLAAPMSGAAVAIWGEDLEDPPSDALLRWSAPHVTAMIETAAS

SEQ ID NO: 11 - protein, 445 aa, Curtobacterium luteum
DTAVSAVTVTKVTASTTGTAVRTTLKVENTAPVRKPASSVWLYLSAGTEKYTLGRVAVKALSAGSSTSVTAVRGTPSRAAAGKYWVLACAGAYSAKQCRTSTATVTTKPTKRARPETGVMLDVARAYYPVALIKRYIDLLADDGGRFLHLHLTDDQNVGIESTVLGQTPANADLDHGVYTSRVTHRPFLSAAQARTISEYGAERGVTIVPEIDTPGHMAAAFALLEAQHGTKWVDRIRSGENELDTSAPESLVLAKKLYAEVQRTFPSSRTVHIGGDEWGDDVTAAHRVAWMNEMAATLGNREVWAWNDGIDRVAVGRLDPRIHVTYWSFDGDTEDAAERRERRARRASAVDLQQAGIDQLNYNSYYLYEVPTDLDPADSDYTVADLRENWSLRAWDGDSGSLLAAPMSGAAVAIWGEDLEDPPSDALLRWSAPHVTAMIETAAS

SEQ ID NO: 12 - protein, 458 aa, Curtobacterium oceanosedimentum
IGGSAGTADASGAPRLVVTKVTASSTTTSTRTTVRTTLTVKNTSVARKPAADAWLSLTAGSKRYTLGHVSVQSLAAGASATIHATHTAPPRAPAGKYAVLACTGAFSLSKCGTSATTVTTARATRARPDTGVMLDVARAYYPVALIEQYIALLADHGGRFLHLHLTDDQNVGIESEVLGQTLANADLRDGVYTSRITGRPFLSAAQAREISRYAAQRGIAIIPEIDTPGHMAAAFALLEAGHGKQWVDRIRSGESELDTSAPGSSALAARLLQEVTRTFPSSRTVHIGGDEWGDDVTADERVQWLNTMAAAVGNRAVWAWNDGIDRAAIGRLDPRIHVTYWSFDGDTEDATERRERRERRAGANDLYAAGIDLLNYNSYYLYEVPTDLDAADSEYTVADLRENWSLRTWDGDSGARLAGPTSGAAVAIWGEDLEAPPSDALLRWSAPHVLAMIETAGS

SEQ ID NO: 13 - protein, 450 aa, Curtobacterium leaf154
AGSTTSTVTVTQVTATTTASSTGTAVRTTLKIKNTAAVRKPASSAWLYLSAGTKKYTLGRVAVKALAAGSSTSVTAVRGTPSRATAGEYSVLACAGAYSAKQCRTSTATVTTKPTKRARPETGVMLDVARAYYPVALIKRYIDLLADDGGRFLHLHLTDDQNVGIESTVLGQTLANADLDEGVYTSRVTRRPFLSAAQARTISDYAARRGVAIVPEIDTPGHMTAAFDLLEAQHGTKWVDRIRSGENELDTSTPGSLALAKKLYAEVQRTFPASRTVHIGGDEWGDDVSAAERVAWMNAMAAALGNREVWAWNDGIDRVAVGRLDPRIHVTYWSFDGDTEDAAERRERRARRASAVDLQQAGIDMLNYNSYYLYEVPTDLDPADSEYTVADLRENWSLRTWDGDSGSLLAAPMSGAAVAIWGEDLEDPPSDALLRWSAPHVTAMIETAAS

SEQ ID NO: 14 - protein, 324 aa, Terribacillus saccharophilus
QDQEKGITIDISRKYYSIKTLKAIVDEISANGGDYLQLHFSDNESYAIASEFLGQNSENPNSAYLTKKELLSLIAYSNDRNIMVIPDIDLPAHSKGWLNIMKEKDSGLYTDIVTDYSEDTLDYHNNAVALYTANQLLDEVLDLFYQPKFAGKQRIVLGGDEVPGSGAHQTDFIRFMNQIAKTAKASNYEPQMWNDSITPEGIQNLDRSFSILYWKQSTLSNGAQSLDVQDFEENGLSVYNYNAYSLYFLPSTRFTQEDITEQIDYMKWAYAYNKFFYISDYYKQVDTPNVKGSSLVFWGEHANDLSQEGLLKQEKPLIQNFLGL

SEQ ID NO: 15 - protein, 324 aa, Terribacillus goriensis
QDQEKGITIDISRKYYSIETLKSIIDEISANGGDYLQLHFSDNERYAIASEFLGQNGENPNSTYLTKKELLSLIAYSNDRDIMVIPDIDLPAHSRGWLNIMKEKDSGLYTDIVTDYSEDTLDYHNNAVALYTANQLLDEVLDLFYQPKFAGKQRIVLGGDEVPGSGVHQTDFIRFMNQIAETAKASNYKPQMWNDSITPEGIQNLDRSFSILYWKQSTLSNGAQGLDVQDFEENGLSVYNYNAYSLYFLPATRFTQEDITEQIDYMKWAYAYNKFFYISDYYKQVDTSNVKGSSLVFWGEHANDLSQEGLLKQEKPLIQNFLGL

SEQ ID NO: 16 - protein, 324 aa, Terribacillus saccharophilus
KDQEKGITIDISRKYYSIGTLKAIVDEINANGGDYLQLHFSDNESYAIASEFLGQNSENPNSTYLTKKELLSLIAYSNDRNIMVIPDIDLPAHSKGWLNVMKEKDSGLYTDIVTDYSEDTLDYHNNAAALYTANQLLDEVLDLFYQPKFAGKQRIVLGGDEVPGSGAHQTDFIRFMNQIDETAKASNYEPQMWNDSITPEGIQNLDRSFSILYWKQSTLSSGAQGLDVQNFEEKGFSVYNYNAYSLYFLPSTRFTQEDITEQIDYMKWAYAYNKFFYISDYYKQVDTSNVKGSSLVFWGEHANDLSQEGLLEQEKPLIQNFLSL

SEQ ID NO: 17 - protein, 324 aa, Terribacillus saccharophilus [sequence omitted]

SEQ ID NO: 18 - protein, 324 aa, Terribacillus saccharophilus
KDQEKGISIDISRKYYSIGTLKAIIDEISANGGDYLQLHFSDNESYAIASDYLGQISDTPNNTYLTKNDLLSLIAYSNDRNILIIPDMDLPAHSRGWLELMKVKDRELYTDIVTDYSNETLDYHNNTDALNTANQLLNEILELFYQPKFAGKQRIVLGGDEVPGSEIHQLDFIRFINQIASTAKASNYAPQMWNDSITAEGIQNLDKSFSILYWKQSTLSNGAQSLEVQDFEDWDFPVYNYNAYSLYFLPSIRFTDEDITEQMNYMKWAYAYNKFFYISDYYKSVDASNVKGSSLTFWGEHATDLSQEELLEQELPLIKKFLSL

SEQ ID NO: 19 - protein, 331 aa, Lactobacillus paraplantarum
NSSTLNTSQGVMLDLGRHPLDETAIKAVISAAAEQHMQYVELHLSDNEHLCFQSAYLGNAASATVLSATTLEQLVAYANQLNIELVPDVDLPSHAGAILRQLQQTHPDIYNTVKLDDETIDYTKPAAISLATTLYGELDASFNNQSQHDLMLGADEVPGSASAYIELTTFINQVSRFQNQHGFNTSIWNDSLLKNELTRLDSNITINYWSQSGNNTDVAIIADRYANRVSVPDILASGHPIVNCNSYATYYQIKNIGNVNDDDYFINYLNHTFRPNIFNEIDTNGHNQDWTIEDGVTTNGILVSLWGADSEHVTPTAIVNFIKRMTIPRSF

SEQ ID NO: 20 - protein, 353 aa, Lactobacillus apinorum
TLADTSNDTKRIGLSLDCSRTYYSPSTIKKYIDLLKKDHGTYLQLHLNDNERYGVESSTLGQTTQNATLKDGVYYNNKTHLAFLSKNQLLDVIQYGYTHGIEVIPEIDLPGHAQSIFKLLSYTSEGKKLVKELENKDGYNEMYYNKQATIDFSKKLLSEYVGMLPSGYHIIVGADEITISDKSDQEAVVKYINAIDDYVNANHLKLEMWNDSFHKAVLSKYHKDILINYWSLTGEVSSSKDRKDNIRMRATLPELNKAGFKTINYNSYYLYMITDPTSFTNESKKIWTSEFKKWKMNMWNDESTKDITKSANNIGAAISIWGEYPNQYTGDQTYNKTYYYVDTFLKAQDKFTK

SEQ ID NO: 21 - protein, 331 aa, Lactobacillus paraplantarum
NSSTLNTSQGVMLDLGRHPLDETAIKAVISAAAEQHMQYVELHLSDNEHLCFQSAYLGNAASATVLSATTLEQLVAYANQLNIELVPDVDLPSHAGAILRQLQQTHPDIYNTVKLDDETIDYTKPAAVSLATTLYGELDASFNNQSQHDLMLGADEVSGSASAYIELTTFINQVSRFQNQNGFNTSIWNDSLLKNELNRLDSNITINYWSQSGNNTDAAIIADRYANRASVPDILASGHPIVNCNSYATYYQFKNIGNVNDDNYFINYLNHTFRPNIFNEIDTNGHNQDWTIEDGVTTNGILVSLWGADSEHVTPTAIVNFIKRMAIPRSF

SEQ ID NO: 22 - protein, 323 aa, Staphylococcus cohnii
QDFQKGINVDIARKDYSLKSLKKIVDTIHENNGDYLQLHFSDNENYAIESQFFKHENIASQNYLSQQELKNLIHYSNKLNIMVVPEFDLPSHSKAWLLLLKNENSNLHENIVSDYSDETIDFFSNQKALEISKRQIKEILNLFHQPNFQKEQRIVLGGDEVPGGKSYQNDFINFMNEIGEYAYQNGYEPQIWNDSITKNGLKLLKNYFSVIFWKQSNNENNEPGITVEDFLDYNFKVYNYNFYSLYFLPSKNYSPTDIEEQTSYISWAYNHNSFYYLKNPYYEVDSLNIQGSALSFWGEHATGMREEEVLNQELPLIRTYLNK

SEQ ID NO: 23 - protein, 321 aa, Staphylococcus fleurettii
ESIQEGVSVDIARKEYSLESLKQIVDTIHENNGQYLQLHFSDDENYAIESDYFSHQGIPNENYLTKAEIKSLIAYSNELNVMVVPDIDFPSHSKALLSLIKNEDKDLYNQIISDYSDNTFDFFSNDKALAISKRHIGEITTLFNQPKYNGQQRIVLGGDEVPGGGAYQSDFISYMNNIGSYAAGQGYEPQMWNDMISHEGIKSLNDTFSILYWKQNENSKSDLTVEDFAEYDFKIYNYNFYSLYFLPSNQFTNADIEEQADYISWAYAYNKFFYTNEPYQEVDSDNVKGSALSFWGEDALNMSQTELINQEIPLIKAYFSS

SEQ ID NO: 24 - protein, 297 aa, Artificial
  Psp4Man4 mature protein sequence
MATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 25 - protein, 10 aa, Artificial
  protein motif
  MISC_FEATURE (2)..(2): X is F or Y
  MISC_FEATURE (7)..(8): X is any amino acid
WXKNDLXXAI

SEQ ID NO: 26 - protein, 12 aa, Artificial
  protein motif
  MISC_FEATURE (3)..(5): X is any amino acid
  MISC_FEATURE (8)..(8): X is any amino acid
  MISC_FEATURE (10)..(10): X is any amino acid
LDXXXGPXGXLT

SEQ ID NO: 27 - protein, 12 aa, Artificial
  protein motif
  MISC_FEATURE (3)..(3): X is M or L
  MISC_FEATURE (4)..(4): X is V or A
  MISC_FEATURE (5)..(5): X is A or T
  MISC_FEATURE (8)..(8): X is N, A or S
  MISC_FEATURE (10)..(10): X is S, T or N
LDXXXGPXGXLT

SEQ ID NO: 28 - protein, 12 aa, Artificial
  protein motif
  MISC_FEATURE (3)..(3): X is L or M
  MISC_FEATURE (8)..(8): X is N or A
  MISC_FEATURE (10)..(10): X is S or T
LDXATGPXGXLT

SEQ ID NO: 29 - protein, 298 aa, Artificial
  PspMan138 mature enzyme sequence
MATGFYVSGNKLYDSTGKEFVMRGVNHGHTWFKNDLNEAIPAIAKTGANTVRIVLSNGVQYTRDDLDAVKNIINVVNQNKMIAVLEVHDATGKDDYDSLDAAINYWISIKEALIGKEDRVIVNIANEWMGTWNGSAWADGYKKAIPKLRNAGIKNTLIVDAAGWGQYPQSIVDYGQSVFAADSLKNTVFSIHMYEYAGKDAATVKANMENVLNKGLALIIGEFGCYHVNGDVDELAIMRYGQELGVGWLAWSWYGNSDGLRYLDMATGPNGSLTSFGNTVVNDTYGIKNTSQKAGIFQ

SEQ ID NO: 30 - protein, 297 aa, Paenibacillus sp.
ATGFYVSGTKLYDSTGKPFVMRGVNHSHTWFKNDLNAAIPAIAKTGANTVRIVLSNGVQYTRDDVNSVKNIISLVNQNKMIAVLEVHDATGKDDYASLDAAINYWISIKDALIGKEDRVIVNIANEWYGTWNGSAWADGYKQAIPKLRNAGIKNTLIVDAAGWGQYPQSIVDYGQSVFAADSLKNTVFSIHMYEYAGGTDAMVKANMEGVLNKGLPLIIGEFGGQHTNGDVDELAIMRYGQQKGVGWLAWSWYGNNSDLSYLDLATGPNGSLTTFGNTVVNDTNGIKATSKKAGIFQ

SEQ ID NO: 31 - protein, 299 aa, Bacillus sp.
NSGFYVSGTTLYDANGNPFVMRGINHGHAWYKDQATTAIEGIANTGANTVRIVLSDGGQWTKDDIQTVRNLISLAEDNNLVAVLEVHDATGYDSIASLNRAVDYWIEMRSALIGKEDTVIINIANEWFGSWDGAAWADGYKQAIPRLRNAGLNNTLMIDAAGWGQFPQSIHDYGREVFNADPQRNTMFSIHMYEYAGGNASQVRTNIDRVLNQDLALVIGEFGHRHTNGDVDESTIMSYSEQRGVGWLAWSWKGNGPEWEYLDLSNDWAGNNLTAWGNTIVNGPYGLRETSKLSTVFTG

SEQ ID NO: 32 - protein, 309 aa, Bacillus sp.
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSP

SEQ ID NO: 33 - protein, 293 aa, Bacillus sp.
GFSVDGNTLYDANGQPFVMRGINHGHAWYKDTASTAIPAIAEQGANTIRIVLSDGGQWEKDDIDTIREVIELAEQNKMVAVVEVHDATGRDSRSDLNRAVDYWIEMKDALIGKEDTVIINIANEWYGSWDGSAWADGYIDVIPKLRDAGLTHTLMVDAAGWGQYPQSIHDYGQDVFNADPLKNTMFSIHMYEYAGGDANTVRSNIDRVIDQDLALVIGEFGHRHTDVDEDTILSYSEETGTGWLAWSWKGNSTSWDYLDLSEDWAGQHLTDWGNRIVHGADGLQETSKPSTVF

SEQ ID NO: 34 - protein, 299 aa, Bacillus sp.
ANSGFYVSGTTLYDANGNPFVMRGINHGHAWYKDQATTAIEGIANTGANTVRIVLSDGGQWTKDDIHTVRNLISLAEDNHLVAVPEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFT

SEQ ID NO: 35 - protein, 309 aa, Bacillus sp.
ANSGFYVSGTTLYDANGNPFVMRGINHGHAWYKDQATTAIEGIANTGANTVRIVLSDGGQWTKDDIHTVRNLISLAEDNHLVAVPEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSP

SEQ ID NO: 36 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 37 - protein, 480 aa, Artificial
  PcuAmy1 mature alpha-amylase polypeptide
ADNGTIMQYFEWYLPNDGAHWNRLNNDAQNLKNVGITAVWIPPAYKGGSSADVGYGVYDTYDLGEFNQKGTVRTKYGTKSELISAVNNLHAKGIAVYGDVVLNHRMNADATELVDAVEVDPNNRNVETTSTYQIQAWTQYDFPGRGNTYSSFKWRWYHFDGVDWDQSRGLNRIYKLRGDGKDWDWEVDSEYGNYDYLMGADLDFNHPDVVNETKTWGKWFVNTVNLDGVRLDAVKHIKFDFMRDWVNNVRSTTGKNLFAVGEYWHYDVNKLNSYITKTNGTMSLFDVPLHFRFYDASNGGGGYDMRNLLNNTLMSSNPMKAVTFVENHDTQPTQALQSTVQSWFKPLAYATILTREQGYPCVFYGDYYGTSDGKISSYKPIMDKLLNARKVYAYGTQRDYFDHPDIVGWTREGDAAHAGSGLATLITDGPGGSKWMYVGTSKAGQVWTDKTGNRSGTVTIDANGWGNFWVNGGSVSVWAK

SEQ ID NO: 38 - protein, 484 aa, Artificial
  Bacillus sp. TS-23 alpha-amylase C-terminally truncated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 39 - protein, 464 aa, Bacillus hemicellulosilyticus
QTHSGFYIEGSTLYDANGEPFVMRGINHGHAWYKHDSNVAIPAIANQGANTIRIVLSDGGQWAKDDINTLNQVLDLAEEHEMIAVVEVHDATGSNSMADLNRAVDYWIEMKDALIGKEDRVIINIANEWYGAWDGQGWANGYKEVIPRLRNAGFTHTLMVDAAGWGQYPQSIHDYGQEVFNADPLANTMFSIHMYEYAGGNASMVQSNIDGVVDQGLALVIGEFGHMHTDGDVDEATILSYSQQRGVGWLAWSWKGNGTQWEYLDLSYDWQGTNLTSWGNTIVHGPNGLLETSIPSSIFHTAPNNGDPPPHNGNETILYDFEHGTQGWSGSSLLGGPWTTNEWSTNGNHSLKADIFLSANSKHELAKVENRNLSGYSTLQATVRHAHWGNVGNLTARMYVKTGSNYSWFNGDPIPVNSANGTTVTLPLSSIPNLNDVKEIGVEFIGASNSNGQTAIYLDHVTIQ

SEQ ID NO: 40 - protein, 289 aa, Bacillus clausii
QNGFHVSGTELLDKNGDPYVMRGVNHGHSWFKQDLEEAIPAIAETGANTVRMVLSNGQQWEKDDASELARVLAATETYGLTTVLEVHDATGSDDPADLEKAVDYWIEMADVLKGTEDRVIINVANEWYGSWRSDVWAEAYAQAIPRLRSAGLSHTLMVDAAGWGQYPASIHERGADVFASDPLKNTMFSIHMYEYAGADRATIAYNIDRVLAENLAVVIGEFGHRHHDGDVDEDAILAYTAERQVGWLAWSWYGNSGGVEYLDLAEGPSGPLTSWGKRIVYGENGLKSD

SEQ ID NO: 41 - protein, 466 aa, Virgibacillus soli
ASGFYVNGNTLYDATGTPFVIRGINHAHSWFKDDTATAIPAIAATGANTIRIVLSDGSQYSRDDIDGVRNLISLAEENNLIAMLEVHDATGKDDISSLDSAADYWISIKEALIGKEDKVLINIANEWYGTWDGASWADGYKQVIPKLRNAGLNHTLIVDSAGWGQFPESIHNYGKEVFNADPLQNTMFSIHMYEYAGGDASTVKANIDGVLNQGLAVIIGEFGHRHTDGDVDEATIMNYSQEKNVGWLAWSWKGNGMEWDYLDLSYDWAGNNLTDWGNTIVNSTNGLKATSEISPVFGDGDDGVGDGGPGDSNGTETTLYNFETGTEGWSGENIETGPWSVNEWAAKGNHSLKADVNLGDNSEHYLYLTQNLNFSGKSQLTATVKHADWGNFGDEINAKLYVKTESDWQWFDGGIEKINSSIGTIITLDLSSLSNPSDIKEVGVQFTGSSNSYGLTALYVDNVTIK